(19) United States
(12) Patent Application Publication
(10) Pub. No.: US 2024/0252472 A1
(43) Pub. Date: Aug. 1, 2024

(54) COMBINATIONS FOR TREATING CANCER

(71) Applicant: Purdue Pharma L.P., Stamford, CT (US)

(72) Inventors: Andrea Díaz-Tejedor, Salamanca (ES); Mercedes Garayoa, Salamanca (ES); Teresa Paíno, Salamanca (ES); Enrique M. Ocio, Salamanca (ES)

(21) Appl. No.: 18/420,883

(22) Filed: Jan. 24, 2024

Related U.S. Application Data
(60) Provisional application No. 63/441,037, filed on Jan. 25, 2023.

(51) Int. Cl. A61K 31/4184; A61K 39/395; A61P 35/00

(52) U.S. Cl. CPC A61K 31/4184; A61K 39/3955; A61P 35/00

(57) ABSTRACT

The present invention relates to the use of tinostamustine or a pharmaceutically acceptable salt thereof in combination with one or more anti-CD38 antibodies in the treatment of cancer.

### CROSS-REFERENCE TO RELATED APPLICATIONS

This application claims priority to and the benefit of the filing date of U.S. Provisional Patent Application No. 63/441,037, filed on Jan. 25, 2023, the entire contents of which are incorporated herein by reference.

### FIELD OF THE INVENTION

The present invention relates to the treatment of cancer, particularly haematological cancer, and more particularly multiple myeloma.

### BACKGROUND OF THE INVENTION

Multiple myeloma (MM) is a cancer of plasma cells. Normal plasma cells are found in the bone marrow and are an important part of the immune system. The immune system provides several different types of cells to fight infection, such as lymphocytes, which includes T cells and B cells. In response to infection, normal plasma cells can generate antibodies, which help the body to attack the infection. The 5-year relative survival rate for patients diagnosed with multiple myeloma is around 60%, relative to healthy individuals. Treatment of multiple myeloma can include surgery, radiation therapy, and chemotherapy (American Cancer Society; cancer.org).

The glycoprotein CD38 has been identified as a promising target in the treatment of cancer, particularly haematological cancers, including multiple myeloma. Anti-CD38 monoclonal antibodies, such as daratumumab (Darzalex) and Isatuximab (Sarclisa) have been clinically approved for use in the treatment of multiple myeloma. More recently, a formulation comprising daratumumab and hyaluronidase (Darzalex Faspro) has been approved for use in the treatment of multiple myeloma. Other anti-CD38 antibodies are in clinical and preclinical development.

Although CD38 may be overexpressed in haematological cancers, it is expressed at low levels in normal cells such as red blood cells (approximately 1000-fold less relative to haematological cancer). Owing to the large number of red blood cells in the body of a patient, treatment with anti-CD38 antibodies can result in off-target binding to surface CD38 in non-cancerous cells. As a result, anti-CD38 antibodies are typically administered at high doses intravenously to achieve therapeutic efficacy. Consequently, the patient may experience adverse side effects due to high doses. There is, therefore, a need to improve the selectivity of anti-CD38 antibodies for cancer cells, and to improve the therapeutic efficacy of anti-CD38 antibodies.

Furthermore, expression of CD38 in a tumor may be heterogeneous. The heterogeneity in the tumor phenotype may lead to relapse following an initial course of treatment, which correlates with poor patient prognosis and outcome. There is, therefore, a need to improve the sensitivity of cancer cells to anti-CD38 antibodies, to avoid instances of patient relapse and refractory disease.

In WO-A-2010/085377, the compound of formula I below is disclosed. The compound of formula I has an INN of tinostamustine and is also known in the art as EDO-S101. The terms “tinostamustine” and “EDO-S101” are used herein interchangeably.

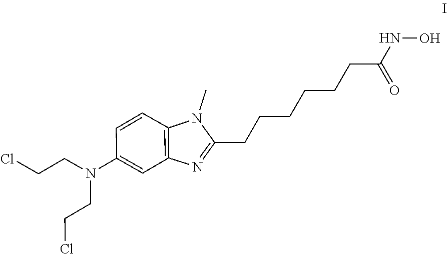 [SMILES: Cn1c(CCCCCCC(=O)NO)nc2cc(N(CCCl)CCCl)ccc21]

Tinostamustine/EDO-S101 is a first-in-class dual-functional alkylating-HDACi fusion molecule which potently inhibits HDAC-regulated pathways. Biological assays showed that tinostamustine potently inhibits HDAC enzyme (HDAC1 IC₅₀ of 9 nM). Tinostamustine an AK-DAC (a first-in-class alkylating deacetylase molecule) that, in preclinical studies, has been shown to simultaneously improve access to the DNA strands within cancer cells, break them and block damage repair.

Existing chemotherapies for cancer may cause patients to experience significant side effects, and in instances may result in refractory disease or recurrence, leading to a poor patient prognosis and outcome (American Cancer Society; cancer.org). There is therefore a need for new and improved therapies in treating cancer, and more particularly therapies that are selective for cancer cells over healthy tissue.

### SUMMARY OF THE INVENTION

One aspect of the invention provides a method of treating cancer in a patient in need thereof, the method comprising administering to the patient a therapeutically effective amount of tinostamustine or a pharmaceutically acceptable salt thereof, in combination with a therapeutically effective amount of one or more anti-CD38 antibod(ies) or an antigen-binding fragment thereof (or collectively “one or more anti-CD38 antibod(ies)” for simplicity), wherein the cancer is capable of expressing CD38 upon contacting tinostamustine or pharmaceutically acceptable salt thereof. In certain embodiments, the cancer is characterized by or is associated with overexpression of CD38 upon contacting tinostamustine or pharmaceutically acceptable salt thereof (e.g., as compared to before contacting tinostamustine or pharmaceutically acceptable salt thereof). In certain embodiments, the cancer has increased expresión of CD38 upon contacting tinostamustine/pharmaceutically acceptable salt thereof, as compared to without contacting tinostamustine/or pharmaceutically acceptable salt thereof.

In certain embodiments, the one or more anti-CD38 antibodies are selected from the group consisting of: daratumumab, isatuximab, MOR202, and combinations thereof.

In certain embodiments, the one or more anti-CD38 antibodies are daratumumab.

In certain embodiments, the cancer is a haematological cancer.

In certain embodiments, the haematological cancer is selected from the group consisting of: leukaemia, lymphoma and multiple myeloma.

In certain embodiments, the haematological cancer is multiple myeloma.

In certain embodiments, tinostamustine or pharmaceutically acceptable salt thereof, and the one or more anti-CD38 antibodies (or antigen-binding fragment thereof), are administered concurrently, sequentially, or separately (with respect to tinostamustine or pharmaceutically acceptable salt thereof).

In certain embodiments, tinostamustine or pharmaceutically acceptable salt thereof, and the one or more anti-CD38 antibodies (or antigen-binding fragment thereof), are administered separately, wherein in some embodiment, tinostamustine or pharmaceutically acceptable salt thereof is administered prior to the one or more anti-CD38 antibodies (or antigen-binding fragment thereof).

In certain embodiments, tinostamustine or pharmaceutically acceptable salt thereof, and the one or more anti-CD38 antibodies (or antigen-binding fragment thereof), are administered concurrently.

In certain embodiments, the molar ratio of tinostamustine or pharmaceutically acceptable salt thereof, and the one or more anti-CD38 antibodies (or antigen-binding fragment thereof), is 1:1000 to 1000:1, 1:700 to 700:1, 1:500 to 500:1, 1:300 to 300:1, 1:100 to 100:1, or 1:50 to 50:1.

In certain embodiments, the expression of CD38 in the cancer or cancer cells is heterogeneous.

In certain embodiments, the expression of CD38 in the cancer or cancer cells is low.

Another aspect of the invention provides a combination comprising (a) tinostamustine or a pharmaceutically acceptable salt thereof, and (2) one or more anti-CD38 antibodies or an antigen-binding fragment thereof.

In certain embodiments, the one or more anti-CD38 antibodies are selected from the group consisting of: daratumumab, isatuximab, MOR202, and combinations thereof.

In certain embodiments, the one or more anti-CD38 antibodies are daratumumab.

In certain embodiments, the molar ratio of Tinostamustine or pharmaceutically acceptable salt thereof, and the one or more anti-CD38 antibodies (or antigen-binding fragment thereof), is 1:1000 to 1000:1, 1:700 to 700:1, 1:500 to 500:1, 1:300 to 300:1, 1:100 to 100:1, or 1:50 to 50:1.

Another aspect of the invention provides a pharmaceutical composition comprising a pharmaceutically acceptable carrier and a combination of the invention.

Another aspect of the invention provides a kit comprising a combination of the invention, or a pharmaceutical composition of the invention.

In certain embodiments, the kit further comprises instructions for treating a cancer in a patient according to the method of the invention.

Another aspect of the invention provides tinostamustine or a pharmaceutically acceptable salt thereof, for use in the treatment of cancer, wherein tinostamustine or pharmaceutically acceptable salt thereof is used in combination with one or more anti-CD38 antibodies or an antigen-binding fragment thereof.

In certain embodiments, the one or more anti-CD38 antibodies are selected from the group consisting of: daratumumab, isatuximab, MOR202, and combinations thereof.

In certain embodiments, the one or more anti-CD38 antibodies are daratumumab.

In certain embodiments, the cancer is a haematological cancer.

In certain embodiments, the haematological cancer is selected from the group consisting of: leukaemia, lymphoma and multiple myeloma.

In certain embodiments, the haematological cancer is multiple myeloma.

In certain embodiments, tinostamustine or a pharmaceutically acceptable salt thereof, and the one or more anti-CD38 antibodies (or antigen-binding fragment thereof), are administered concurrently, sequentially, or separately.

In certain embodiments, tinostamustine or a pharmaceutically acceptable salt thereof, and the one or more anti-CD38 antibodies (or antigen-binding fragment thereof), are administered separately, wherein tinostamustine or pharmaceutically acceptable salt thereof is in some embodiments administered prior to the one or more anti-CD38 antibodies (or antigen-binding fragment thereof).

In certain embodiments, tinostamustine or pharmaceutically acceptable salt thereof, and the one or more anti-CD38 antibodies (or antigen-binding fragment thereof), are administered concurrently.

In certain embodiments, the molar ratio of tinostamustine or a pharmaceutically acceptable salt thereof and the one or more anti-CD38 antibodies (or antigen-binding fragment thereof) is 1:1000 to 1000:1, 1:700 to 700:1, 1:500 to 500:1, 1:300 to 300:1, 1:100 to 100:1, or 1:50 to 50:1.

Another aspect of the invention provides an anti-CD38 antibody or an antigen-binding fragment thereof, for use in the treatment of cancer, wherein the anti-CD38 antibody or antigen-binding fragment thereof is used in combination with tinostamustine or a pharmaceutically acceptable salt thereof.

In certain embodiments, the use is in accordance with any one of the methods of the invention.

In certain embodiments, the tinostamustine or pharmaceutically acceptable salt thereof, and an anti-CD38 antibody (or antigen-binding fragment thereof), are for use in the treatment of cancer.

In certain embodiments, the tinostamustine or pharmaceutically acceptable salt thereof, and an anti-CD38 antibody (or an antigen-binding fragment thereof), are for use according to the methods of the invention.

Another aspect of the invention provides a combination of tinostamustine or a pharmaceutically acceptable salt thereof, and one or more anti-CD38 antibodies or an antigen-binding fragment thereof.

In certain embodiments, the one or more anti-CD38 antibodies are selected from the group consisting of: daratumumab, isatuximab, MOR202, and combinations thereof.

In certain embodiments, the one or more anti-CD38 antibodies are daratumumab.

In certain embodiments, the combination is for use as a medicament.

In certain embodiments, the combination is for use in the treatment of cancer.

In certain embodiments, the cancer is a haematological cancer, optionally wherein the haematological cancer is selected from the group consisting of: leukaemia, lymphoma and multiple myeloma.

In certain embodiments, the haematological cancer is multiple myeloma.

In certain embodiments, tinostamustine or pharmaceutically acceptable salt thereof, and the one or more anti-CD38 antibodies (or antigen-binding fragment thereof), are administered concurrently, sequentially, or separately.

In certain embodiments, tinostamustine or pharmaceutically acceptable salt thereof, and the one or more anti-CD38 antibodies (or antigen-binding fragment thereof), are administered separately, wherein tinostamustine or pharmaceutically acceptable salt thereof is, in some embodiments, administered prior to the one or more anti-CD38 antibodies (or antigen-binding fragment thereof).

In certain embodiments, tinostamustine or pharmaceutically acceptable salt thereof, and the one or more anti-CD38 antibodies (or antigen-binding fragment thereof), are administered concurrently.

In certain embodiments, the molar ratio of Tinostamustine or pharmaceutically acceptable salt thereof and the one or more anti-CD38 antibodies is 1:1000 to 1000:1, 1:700 to 700:1, 1:500 to 500:1, 1:300 to 300:1, 1:100 to 100:1, or 1:50 to 50:1.

Another aspect of the invention provides a pharmaceutical composition comprising a pharmaceutically acceptable carrier and a combination of the invention.

Another aspect of the invention provides a kit comprising a combination of the invention or a pharmaceutical composition of the invention.

In certain embodiments, the kit further comprises instructions for treating a patient; said instructions optionally providing instructions for the use of combination(s) of the invention.

Another aspect of the invention provides a use of tinostamustine or a pharmaceutically acceptable salt thereof in the manufacture of a medicament for the treatment of cancer, wherein in said treatment Tinostamustine or pharmaceutically acceptable salt thereof is administered in combination with one or more anti-CD38 antibodies or an antigen-binding fragment thereof.

In certain embodiments, the one or more anti-CD38 antibodies is administered in combination with tinostamustine or a pharmaceutically acceptable salt thereof.

In certain embodiments, tinostamustine or a pharmaceutically acceptable salt thereof and the one or more anti-CD38 antibodies (or antigen-binding fragment thereof) are administered in combination.

### BRIEF DESCRIPTION OF THE DRAWINGS

The present invention is described in further detail below with reference to the accompanying Figures in which:

FIG. 1A illustrates normalized mean fluorescence intensity (MFI) for expression of CD38 in MM cells (MM1.S, JJN3, KMS12-BM, RPMI-8226, U266, MOLP-8) after treatment with a control (0 μM EDO-S101, DMSO) or EDO-S101 (0.1, 0.5, 1 and 2.5 μM).

FIG. 1B illustrates flow cytometry histograms showing CD38 expression in MM cells (JJN3, KMS12-BM, RPMI-8226, MOLP-8) after treatment with a control (0 M EDO-S101, DMSO) or EDO-S101 (0.1, 0.5, 1 and 2.5 M).

FIG. 2A illustrates the percentage of CD38+ cells in a population of MM cells (JJN3 and U226) after treatment with a control (0 μM EDO-S101, DMSO) or EDO-S101 (1 and 2.5 μM).

FIG. 2B illustrates flow cytometry histograms showing CD38 expression in a population of MM cells (JJN3, U266) after treatment with a control (0 μM EDO-S101, DMSO) or EDO-S101 (2.5 μM).

FIG. 2C shows the percentage of the viable MM cells (U266, JJN3, MM.1S, NCI-H929, RPMI-8226, KMS12-BM, and MOLP-8) after treatment with 1 or 2.5 μM tinostamustine or vehicle (DMSO).

FIG. 3A illustrates a section of a western blot showing expression of CD38 in MM cells (MM1.S, JJN3, RPMI-8226, MOLP-8) after treatment with a control (0 μM EDO-S101, DMSO) or EDO-S101 (1 or 2.5 μm).

FIG. 3B illustrates immunofluorescence images showing expression of CD38 in MM cells (RPMI-8226 and MOLP-8) after treatment with a control (0 μM EDO-S101, DMSO) or EDO-S101 (2.5 μm). DAPI is provided to show cell nuclei.

FIG. 3C shows CD38 mRNA levels in JJN3, MM1.S, RPMI-8226 and MOLP-8 cell lines by qPCR, obtained after incubation with tinostamustine at 1 and 2.5 μM for 36 hours (except for the MOLP-8 cell line that was treated for 6 hours). The results are shown as the fold change between tinostamustine-treated and DMSO-treated cells after normalization with GAPDH and correspond to the average of three experiments.

FIG. 3D shows normalized MFI expression of CD38 on myeloma cells from patients after ex vivo treatment with tinostamustine (1 and 2.5 M) for 48 hours. CD38 MFI for each sample was normalized to that of anti-CD38-APC isotype control.

FIG. 3E shows results of Chromatin Immunoprecipitation (ChIP) signal of CD38 Histone 3 acetylation. MM.1S and RPMI-8226 cell lines were cultured in the presence or absence of tinostamustine (2.5 μM) for 48 hours and immunoprecipitated using anti-acetyl histone H3 antibody (AcH3) or an anti-IgG as a negative control. The GAPDH gene was used as a positive control for AcH3 whereas SAT2 gene was used as a negative control. The RT-qPCR signal relative to the ChIP input is shown.

FIG. 4 illustrates the expression of CD38 mRNA as determined by quantitative PCR in MM cells (MM1.S, JJN3, RPMI-8226) treated with a control (0 μM EDO-S101, DMSO) or EDO-S101 (1 or 2.5 μm).

FIG. 5 illustrates a section of a western blot showing expression of various transcription factors in MM cells (MM1.S, JJN3, RPMI-8226 and MOLP-8) after treatment with a control (0 μM EDO-S101, DMSO) or EDO-S101 (1 or 2.5 μm).

FIG. 6 illustrates normalized mean fluorescence intensity (MFI) for binding of daratumumab to CD38 expressed on MM cells (JJN3, MM1.S, RPMI-8226, MOLP-8) treated with a control (0 μM EDO-S101, DMSO) or EDO-S101 (1 or 2.5 μm).

FIG. 7A illustrates normalized mean fluorescence intensity (MFI) for expression of MICA and MICB on MM cells (JJN3, MM1.S, RPMI-8226, MOLP-8) treated with a control (0 μM EDO-S101, DMSO) or EDO-S101 (1 or 2.5 μm).

FIG. 7B shows mRNA levels of MICA and MICB after treatment with tinostamustine (1 and 2.5 μM) for 36 hours except for the MOLP-8 cell line that was treated for 6 hours. The expression levels for MICA and MICB were normalized to GAPDH levels and fold induction of gene expression was referred to that in absence of tinostamustine considered as 1 (n=3).

FIG. 7C shows MFI expression of MICA and MICB normalized to that of isotype control on myeloma cells from patients after ex vivo treatment with tinostamustine (1 and 2.5 M) for 48 hours.

FIG. 8A illustrates the proportion of a population of MM cells (MOLP-8) determined to have undergone daratumumab-mediated cross-linking mediated apoptosis following treatment with daratumumab (1 μg/mL). The MM cells were pre-treated with a control (0 HM EDO-S101, DMSO) or EDO-S101 (0.1, 0.5, 1 or 2.5 μm).

FIG. 8B shows assessment of the effect of tinostamustine in daratumumab-mediated myeloma cell death in vitro—effect of pre-treatment with tinostamustine on daratumumab-mediated ADCC. MM cell lines were incubated for 48 hours with DMSO or tinostamustine (2.5 μM). After the incubation time, tinostamustine or DMSO were removed from the culture medium and the corresponding mechanism of action was assessed. Tinostamustine or DMSO-pretreated myeloma cell lines were co-cultured with NK cells (ratio 1:1) and further incubated for 4 hours with daratumumab (1 μg/ml) or the isotype control. The percentage of dead cells was analyzed by flow cytometry.

FIG. 9 illustrates the proportion of a population of MM cells (MOLP-8) determined to have undergone daratumumab-mediated complement dependent cytotoxicity (CDC) following treatment treated with daratumumab (0.1 or 1 μg/mL). The MM cells were pre-treated with a control (0 M EDO-S101, DMSO) or EDO-S101 (2.5 μm).

FIG. 10 illustrates the proportion of populations of MM cells (MM1.S, RPMI-8226), MOLP-8), MM cells co-cultured with NK cells, and MM cells co-cultured with NK cells and daratumumab (1 μg/mL) determined to have undergone Antibody-Dependent Cellular Cytotoxicity (ADCC). The MM cells were pre-treated with a control (0 μM EDO-S101, DMSO) or EDO-S101 (2.5 μm).

FIG. 11 illustrates the number of macrophages in a co-culture of macrophages and MM cells that were determined to have phagocytosed MM cells (MOLP-8) by means of Antibody-Dependent Cellular Phagocytosis (ADCP), following treatment with daratumumab (0.1 and 1 μg/mL). FIG. 11 also shows the number of eliminated MM cells. The MM cells were pre-treated with a control (0 μM EDO-S101, DMSO) or EDO-S101 (2.5 μm).

FIG. 12A illustrates the proportion of MM cells determined to have been eliminated following treatment with daratumumab (10 μg/mL), EDO-S101 (0.5, 1 and 2.5 μm) and a combination thereof. MM cells were isolated from patient bone marrow and cultured ex vivo.

FIGS. 12B and 12C show the effect of the combination of tinostamustine+daratumumab in ex vivo cultures of bone marrow samples from MM patients. The cells were incubated for 24 hours with the individual treatments or the combination. The percentage of eliminated events was calculated as explained in Material and methods section for myeloma cells (FIG. 12B) and lymphocytes (FIG. 12C). Each box plot shows data from 10 patients' samples. Statistically significant differences were evaluated by Friedman's test and Wilcoxon post hoc tests (*p<0.05 and **p<0.01).

FIG. 13 illustrates cumulative survival of tumor-bearing (MM1S-Luc) CB17-SCID mice treated with a control, EDO-S101, daratumumab, or a combination of EDO-S101 and daratumumab.

FIG. 14A illustrates tumor volume over time for human plasmacytoma tumor-bearing NSG mice treated with a control, EDO-S101, Daratumumab, or a combination of EDO-S101 and daratumumab.

FIG. 14B illustrates cumulative survival of human plasmacytoma tumor-bearing NSG mice treated with a control, EDO-S101, Daratumumab, or a combination of EDO-S101 and daratumumab.

FIG. 15A illustrates tumor volume over time for human plasmacytoma tumor-bearing CB17-SCID mice treated with a control, EDO-S101, Daratumumab, or a combination of EDO-S101 and daratumumab.

FIG. 15B illustrates cumulative survival of human plasmacytoma tumor-bearing CB17-SCID mice treated with a control, EDO-S101, daratumumab, or a combination of EDO-S101 and daratumumab.

FIG. 16 illustrates effects of treatments administration on body weight of mice, which was monitored throughout the treatment period; and the percentage of weight at each time point was calculated considering day 1 as 100%. X-axes express the time in days from the beginning of the treatment.

### DETAILED DESCRIPTION OF THE INVENTION

The present invention describes a novel treatment of cancer with tinostamustine or a pharmaceutical salt thereof, in combination with one or more anti-CD38 antibodies, partly based on the surprising discovery that tinostamustine up-regulates the expression of CD38 in certain cancer cells (such as multiple myeloma or MM cells), as well as certain cell surface ligands that activates innate immune system effector cells such as NK cells, the latter of which further enhances several anti-CD38 antibody-mediated cancer killing effects including ADCC, CDC, and ADCP. Thus, the combination therapy using tinostamustine and anti-CD38 antibodies has a synergistic effect compared to both monotherapies alone against the same cancer (e.g., MM).

That is, the present invention represents a novel combination for the treatment of cancer, and more particularly for the treatment of haematological cancer, that surprisingly provides improved efficacy in treating cancers relative to tinostamustine or anti-CD38 antibodies as monotherapies.

Although the present invention is described with reference to treatment of multiple myeloma, those skilled in the art will appreciate that the benefits of the present invention may be realized in other cancers characterised by expression of CD38 (e.g., cancers that already express CD38, or cancers that can be induced to express CD38 upon tinostamustine treatment), which would also benefit from a therapy targeting CD38 expressing cancer cells. For example, it is envisaged that the benefits of the present invention may be realized in the treatment of such haematological cancers, including leukaemia, lymphomas and multiple myeloma.

As described in Example 1 it has been surprisingly found that tinostamustine increases the surface expression of CD38 in tumor cells, specifically multiple myeloma (MM) cells. As further described in Example 2, it has further been found that this result can be leveraged to increase the efficacy of an anti-CD38 therapeutic such as daratumumab (an anti-CD38 monoclonal antibody). As described in Examples 4 and 5, treatment of MM cells with tinostamustine in combination with an anti-CD38 antibody in vitro and ex vivo increases the cytotoxicity of the anti-CD38 antibody relative to MM cells not treated with tinostamustine. Further still, as described in Example 6, in vivo studies in a murine model revealed that treatment of MM tumors with a combination of tinostamustine and an anti-CD38 antibody significantly delayed tumor growth and improved overall survival, relative to control animals treated with either tinostamustine or an anti-CD38 antibody as monotherapies.

As such, it has been surprisingly found that tinostamustine can be used to increase the therapeutic efficacy of anti-CD38 antibodies. In certain embodiments, tinostamustine is administered to a patient in need thereof separately from and prior to, administration the anti-CD38 antibody. Alternatively, tinostamustine may be administered to a patient in need concurrently with an anti-CD38 antibody.

Suitable dosage regimens in accordance with the present invention are described in more detail below.

Moreover, as described in Example 3 it has been surprisingly discovered that tinostamustine increases the immunogenicity of tumor cells. Treatment of MM cells with tinostamustine increases the expression on MM cells of ligands for natural killer (NK) cell activating receptors. As such, when used in combination with an anti-CD8 antibody, tinostamustine increases sensitivity to NK-mediated cell death through both direct NK attack by increasing expression of NK cell activating ligands and indirectly through increasing expression of CD38, which in turn leads to antibody-dependent cellular cytotoxicity (ADCC) mediated by NK cells. In other words, both an increase in CD38 expression and an increase in expression of NK cell activating ligands leads to increased sensitivity to NK cell attack and consequently tumor cell death.

The present invention therefore also describes that tinostamustine has dual activity in increasing therapeutic efficacy of anti-CD38 antibodies and in sensitising tumor cells to NK cells. Thus in another aspect, the invention provides a method of enhancing an NK cell-mediated treatment of cancer in a patient in need thereof, the method comprising administering to the patient a therapeutically effective amount of tinostamustine or a pharmaceutically acceptable salt thereof, in order to enhance the expression of a ligand that activates NK cells. In certain embodiments, the NK cell-mediated treatment of cancer comprises ADCC, CDC, and/or ADCP.

Thus one aspect of the invention provides a method of treating cancer in a patient in need thereof, the method comprising administering to the patient a therapeutically effective amount of tinostamustine or a pharmaceutically acceptable salt thereof, in combination with a therapeutically effective amount of an anti-CD38 antibody, wherein the cancer is capable of expressing CD38 upon contacting tinostamustine or pharmaceutically acceptable salt thereof.

In a related aspect, the invention provides tinostamustine or a pharmaceutically acceptable salt thereof, for use in the treatment of cancer, wherein tinostamustine or a pharmaceutically acceptable salt thereof is used in combination with one or more anti-CD38 antibodies, and wherein the cancer is capable of expressing CD38 upon contacting tinostamustine or pharmaceutically acceptable salt thereof.

In a further related aspect of the invention, there is provided an anti-CD38 antibody, for use in the treatment of cancer, wherein the anti-CD38 antibody is used in combination with tinostamustine or a pharmaceutically acceptable salt thereof, and wherein the cancer is capable of expressing CD38 upon contacting tinostamustine or pharmaceutically acceptable salt thereof.

In a further aspect of the invention, there is provided tinostamustine or a pharmaceutically acceptable salt thereof, and an anti-CD38 antibody, for use in the treatment of cancer, wherein the cancer is capable of expressing CD38 upon contacting tinostamustine or pharmaceutically acceptable salt thereof.

In a further aspect of the invention, there is provided a combination of tinostamustine or a pharmaceutically acceptable salt thereof, and one or more anti-CD38 antibodies.

In certain embodiments, the one or more anti-CD38 antibodies is selected from daratumumab, isatuximab and MOR202, or combinations thereof. In certain embodiments, the one or more anti-CD38 antibodies is daratumumab.

In certain embodiments, the cancer to be treated is a haematological cancer. In certain embodiments, the haematological cancer is selected from leukaemia, lymphoma and multiple myeloma. In certain embodiments, the haematological cancer is multiple myeloma.

In certain embodiments, tinostamustine or a pharmaceutically acceptable salt thereof and the one or more anti-CD38 antibodies are administered concurrently, sequentially, or separately.

In certain embodiments, tinostamustine or a pharmaceutically acceptable salt thereof and the one or more anti-CD38 antibodies are administered separately, wherein tinostamustine or a pharmaceutically acceptable salt thereof is administered prior to the one or more anti-CD38 antibodies.

In certain embodiments, tinostamustine or a pharmaceutically acceptable salt thereof and the one or more anti-CD38 antibodies are administered concurrently.

In certain embodiments, the molar ratio of tinostamustine or a pharmaceutically acceptable salt thereof and the one or more anti-CD38 antibodies is from 1:1000 to 1000:1, 1:700 to 700:1, 1:500 to 500:1, 1:300 to 300:1, 1:100 to 100:1, or 1:50 to 50:1.

In certain embodiments, the expression of CD38 in the cancer or cancer cells is heterogeneous.

In certain embodiments, the expression of CD38 in the cancer or cancer cells is low.

In certain embodiments, the method or use delays disease progression. In certain embodiments, the method or use increases disease progression-free survival.

In certain embodiments, the combination is provided for use as a medicament. In certain embodiments, the combination is provided for use as a medicament in the treatment of cancer. In certain embodiments, the cancer is haematological cancer, wherein in some embodiments, the haematological cancer is selected from leukaemia, lymphoma and multiple myeloma. In certain embodiments, the haematological cancer is multiple myeloma.

In certain embodiments, tinostamustine or a pharmaceutically acceptable salt thereof and the one or more anti-CD38 antibodies are administered concurrently, sequentially, or separately. In certain embodiments, tinostamustine or a pharmaceutically acceptable salt thereof and the one or more anti-CD38 antibodies are administered separately, wherein tinostamustine or a pharmaceutically acceptable salt thereof is administered prior to the one or more anti-CD38 antibodies. In certain embodiments, tinostamustine or a pharmaceutically acceptable salt thereof and the one or more anti-CD38 antibodies are administered concurrently.

According to a further aspect of the invention, there is provided a pharmaceutical composition comprising a pharmaceutically acceptable carrier and a combination as defined herein.

According to a further aspect of the invention, there is provided a kit comprising a combination as defined herein. The kit may further include instructions for treating a patient; with said instructions optionally providing instructions for the use of combination(s).

According to a further aspect of the invention, there is provided the use of tinostamustine or a pharmaceutically acceptable salt thereof in the manufacture of a medicament for the treatment of cancer, wherein the cancer is capable of expressing CD38 upon contacting tinostamustine or pharmaceutically acceptable salt thereof, and wherein in said treatment tinostamustine or a pharmaceutically acceptable salt thereof is administered in combination with one or more anti-CD38 antibodies.

According to a further aspect of the invention, there is provided the use of one or more anti-CD38 antibodies in the manufacture of a medicament for the treatment of cancer, wherein the cancer is capable of expressing CD38 upon contacting tinostamustine or pharmaceutically acceptable salt thereof, and wherein said one or more anti-CD38 antibodies are administered in combination with tinostamustine or a pharmaceutically acceptable salt thereof.

According to a further aspect of the invention, there is provided the use of tinostamustine or a pharmaceutically acceptable salt thereof and one or more anti-CD38 antibodies in the manufacture of a medicament for the treatment of cancer, wherein the cancer is capable of expressing CD38 upon contacting tinostamustine or pharmaceutically acceptable salt thereof, and wherein tinostamustine or a pharmaceutically acceptable salt thereof and the one or more anti-CD38 antibodies are administered in combination.

According to a further aspect of the invention, there is provided a method of slowing disease progression in a mammal, wherein the disease is cancer, the method comprising administering to a patient in need thereof a combination of tinostamustine or a pharmaceutically acceptable salt thereof, and one or more anti-CD38 antibodies, and wherein the cancer is capable of expressing CD38 upon contacting tinostamustine or pharmaceutically acceptable salt thereof.

According to a further aspect of the invention, there is provided a method for increasing progression-free survival in a mammal suffering from cancer, the method comprising administering to a patient in need thereof a combination of tinostamustine or a pharmaceutically acceptable salt thereof, and one or more anti-CD38 antibodies, and wherein the cancer is capable of expressing CD38 upon contacting tinostamustine or pharmaceutically acceptable salt thereof.

The present invention will now be further described. In the following passages, different aspects of the invention are defined in more detail. Each aspect so defined may be combined with any other aspect or aspects unless clearly indicated to the contrary. In particular, any feature (such as an advantageous feature) may be combined with any other feature or features (such as another advantageous feature).

### 2. Definitions

In the present application, a number of general terms and phrases are used, which should be interpreted as follows.

The term “treating”, as used herein, unless otherwise indicated, means reversing, attenuating, alleviating or inhibiting the progress of the disease or condition to which such term applies, or one or more symptoms of such disorder or condition. The term “treatment”, as used herein, unless otherwise indicated, refers to the act of treating as “treating” is defined immediately above. The term treating as used herein may also include prophylactic or adjuvant treatment, that is treatment designed to prevent the condition from occurring or minimize the likelihood of the condition occurring. Adjuvant therapy is additional cancer treatment given after the primary treatment to lower the risk that the cancer will come back. Treatment may be a delay in disease progression. Treatment may be an increase in overall survival.

“Patient” includes humans, non-human mammals (e.g., dogs, cats, rabbits, cattle, horses, sheep, goats, swine, deer, and the like) and non-mammals (e.g., birds, and the like).

Tinostamustine, also known as EDO-S101, is a compound of formula I below. The IUPAC name is 7-(5-(bis(2-chloroethyl)amino)-1-methyl-1H-benzo[d]imidazol-2-yl)-N-hydroxyheptanamide.

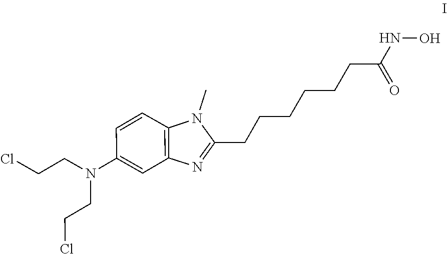 [SMILES: Cn1c(CCCCCCC(=O)NO)nc2cc(N(CCCl)CCCl)ccc21]

Tinostamustine is a first-in-class alkylating deacetylase inhibiting molecule, in clinical development for a range of rare and difficult-to-treat blood cancers and advanced solid tumors. Preclinical studies have shown that tinostamustine has the potential to improve access to the DNA strands within cancer cells, break them and counteract damage repair. The preclinical data also suggests that these complementary and simultaneous modes of action have the potential to overcome resistance towards some other cancer treatments (see for example López-Iglesias et al The Alkylating Histone Deacetylase Inhibitor Fusion Molecule EDO-S101 Displays Full Bi-Functional Properties in Preclinical Models of Hematological Malignancies Blood 2014; 124(21)).

Tinostamustine for use in accordance with the present invention may be prepared following the synthetic process such as the one disclosed in WO-A-2010/085377, which is incorporated herein by reference.

The term “pharmaceutically acceptable salts” refers to any salt which, upon administration to the patient is capable of providing (directly or indirectly) compounds in accordance with the present invention. It will be appreciated that non-pharmaceutically acceptable salts also fall within the scope of the invention since those may be useful in the preparation of pharmaceutically acceptable salts. The preparation of salts can be carried out by methods known in the art.

“Pharmaceutically acceptable salts” means salts of compounds of the present invention which are pharmaceutically acceptable, as defined above, and which possess the desired pharmacological activity. Such salts include acid addition salts formed with inorganic acids, or with organic acids. Pharmaceutically acceptable salts also include base addition salts which may be formed when acidic protons present are capable of reacting with inorganic or organic bases. Generally, such salts are, for example, prepared by reacting the free acid or base forms of these compounds with a stoichiometric amount of the appropriate base or acid in water or in an organic solvent or in a mixture of the two. Generally, non-aqueous media like ether, ethyl acetate, ethanol, isopropanol or acetonitrile can be used. Examples of the acid addition salts include mineral acid addition salts such as, for example, hydrochloride, hydrobromide, hydroiodide, sulfate, bisulfate, sulfamate, nitrate, phosphate, and organic acid addition salts such as, for example, acetate, trifluoroacetate, maleate, fumarate, citrate, oxalate, succinate, tartrate, salicylate, tosylate, lactate, naphthalenesulphonae, malate, mandelate, methanesulfonate and p-toluenesulfonate. Examples of the alkali addition salts include inorganic salts such as, for example, sodium, potassium, calcium and ammonium salts, and organic alkali salts such as, for example, ethylenediamine, ethanolamine, N,N-dialkylenethanolamine, triethanolamine and basic amino acids salts.

In the present invention, the pharmaceutically acceptable salt of tinostamustine can be, for example, hydrochloride, hydrobromide, hydroiodide, sulfate, bisulfate, sulfamate, nitrate, phosphate, citrate, methanesulfonate, trifluoroacetate, glutamate, glucuronate, glutarate, malate, maleate, oxalate, succinate, fumarate, tartrate, tosylate, mandelate, salicylate, lactate, p-toluenesulfonate, naphthalenesulfonate or acetate salt.

Any compound that is a prodrug of tinostamustine is within the scope and spirit of the invention. By way of example, the term “prodrug” is used in its broadest sense and encompasses those derivatives that are converted in vivo to Tinostamustine. The prodrug can hydrolyze, oxidize, or otherwise react under biological conditions to provide tinostamustine. Examples of prodrugs include, but are not limited to, derivatives and metabolites of tinostamustine that include biohydrolyzable moieties such as biohydrolyzable amides, biohydrolyzable esters, biohydrolyzable carbamates, biohydrolyzable carbonates, biohydrolyzable ureides, and biohydrolyzable phosphate analogues. Prodrugs can typically be prepared using well-known methods, such as those described by Burger in “Medicinal Chemistry and Drug Discovery” 6^{th} ed. (Donald J. Abraham ed., 2001, Wiley) and “Design and Applications of Prodrugs” (H. Bundgaard ed., 1985, Harwood Academic Publishers).

In addition, tinostamustine or pharmaceutically acceptable salts as referred to herein may be in crystalline or amorphous form, either as free compounds or as solvates (e.g. hydrates) and it is intended that all forms are within the scope of the present invention. Methods of solvation are generally known within the art.

CD38, also known as cyclic ADP ribose hydroxylase, is a type II transmembrane glycoprotein. CD38 belongs to a family of membrane bound or soluble enzymes capable of converting NAD to cyclic ADP ribose or nicotinic acid-adenine dinucleotide phosphate. CD38 is also an important molecule in cell signalling, cell adhesion, signal transduction and calcium signalling. CD38 may function as either a receptor or an enzyme.

Overexpression of CD38 has been associated with haematological cancers, including multiple myeloma (Lockhorst et al New England Journal of Medicine 2015; 373(13), 1207). Moreover, CD38 has also been used as a prognostic marker in leukaemia and is associated with increased disease progression (Burgler et al; Crit Rev Immunol. 2015; 35(5), 417). CD38 has also been associated with disease progression in other cancers such as lung cancer (Bu et al Carcinogenesis 2018; 39(2), 242)

CD38 has therefore been identified as a promising target in cancer therapy.

The clinical use of CD38 antibodies has been found effictivein treating cancers that express CD38, such as multiple myeloma, and in particular in patients where standard MM therapy has failed. CD38 antibodies have a number of cytotoxic effects on cells. As well as the classical effector mechanisms associated with antibody therapy, CD38 antibodies also induce apoptosis, complement-dependent cytotoxicity (CDC), antibody-dependent cellular cytotoxicity (ADCC) and antibody-dependent cellular phagocytosis (ADCP).

Examples of clinically approved anti-CD38 antibodies for the treatment of haemotaological cancer include isatuximab, daratumumab and MOR202. Both isatuximab and daratumumab are monoclonal antibodies.

Daratumumab has been clinically approved for treatment of multiple myeloma, diffuse large B cell lymphoma, follicular lymphoma, and mantle cell lymphoma (WHO Drug Information). Daratumumab is generally administered intravenously. Daratumumab causes cancer cells to undergo apoptosis via antibody-dependent cellular cytotoxicity (ADCC), complement dependent cytotoxicity (CDC), antibody-dependent cellular phagocytosis (ADCP) or by cross-linking mediated apoptosis.

Isatuximab has been clinically approved for treatment of multiple myeloma, and in particular is intended for treatment of relapsed or refractory multiple myeloma. Isatuximab is undergoing further clinical trials for the treatment of multiple myeloma, and is also undergoing Phase II clinical trials for the treatment of T-cell leukaemia. Isatuximab is generally administered intravenously. Isatuximab has been shown to have more potent inhibition of CD38, relative to daratumumab. Isatuxumab has also been shown to cause increased induction of cell apoptosis.

Felzartamab or MOR202 is a human HuCAL monoclonal anti-CD38 antibody. Binding of MOR202 to CD38 causes ADCC and ADCP-mediated cell killing. MOR202 is currently under clinical investigation in patients with anti-PLA2R antibody-positive membranous nephropathy, a kidney specific autoimmune disease, and in patients with multiple myeloma.

It has been surprisingly shown for the first time that a combination of tinostamustine with one or more anti-CD38 antibodies is efficacious in the treatment of cancer, particularly haematological cancer, and more particularly multiple myeloma.

In accordance with the present invention, tinostamustine and one or more anti-CD38 antibodies are provided for use in the treatment of cancer, and in embodiments haematological cancer. Haematological cancers may include lymphoma, leukaemia and multiple myeloma. Lymphomas may be selected from diffuse large B cell lymphoma, Hodgkin's lymphoma, non-Hodgkin's lymphoma, follicular lymphoma, and mantle cell lymphoma.

In certain embodiments, the haematological cancer is multiple myeloma. The multiple myeloma may be selected from active myeloma, plasmacytoma, light chain myeloma or non-secretory myeloma.

In certain embodiments, the cancer to be treated is refractory. In certain embodiments, the cancer to be treated is relapsed. Patients with refractory disease are categorised by tumor phenotypes that do not respond to existing therapies, such as anti-CD38 antibodies, proteasome inhibitors, and the like. Although some patients may show a positive response to an initial course of treatment, tumor cells may evade the chosen therapy, resulting in recurrence of the cancer (i.e. relapse). The present invention may therefore advantageously be used to treat patients with refractory cancer, by increasing the sensitivity of the cancer cells to treatment with anti-CD38 antibodies, by administration of tinostamustine or a pharmaceutically acceptable salt thereof. The present invention may therefore advantageously be used to treat patients with refractory cancer, by increasing the sensitivity of the cancer cells to treatment with anti-CD38 antibodies, by administration of tinostamustine or a pharmaceutically acceptable salt thereof.

The term “antibody” in the context of the present invention refers to any immunoglobulin, for example, a full-length immunoglobulin. In certain embodiments, the term covers monoclonal antibodies, polyclonal antibodies, multispecific antibodies, such as bispecific antibodies, and antibody fragments thereof, so long as they exhibit the desired biological activity as described above. Antibodies may be derived from any species, such as those of mouse, human or rabbit origin. Alternatively, the antibodies, such as monoclonal antibodies, may be humanised, chimeric or antibody fragments thereof. The term ‘chimeric antibodies’ may also include “primatised” antibodies comprising variable domain antigen-binding sequences derived from a non-human primate (e.g., Old World Monkey, Ape etc) and human constant region sequences. The immunoglobulins can also be of any type (e.g. IgG, IgE, IgM, IgD, and IgA), class (e.g., IgG1, IgG2, IgG3, IgG4, IgA1 and IgA2) or subclass of immunoglobulin molecule.

The term “monoclonal antibody” refers to a substantially homogenous population of antibody molecules (i.e. the individual antibodies comprising the population are identical except for possible naturally occurring mutations that may be present in minor amounts), produced by a single clone of B lineage cells, often a hybridoma. Importantly, each monoclonal has the same antigenic specificity—i.e. it is directed against a single determinant on the antigen.

The production of monoclonal antibodies can be carried out by methods known in the art. However, as an example, the monoclonal antibodies can be made by the hybridoma method (Kohler et al (1975) Nature 256:495), the human B cell hybridoma technique (Kozbor et al., 1983, Immunology Today 4: 72), or the EBV-hybridoma technique (Cole et al., 1985, Monoclonal Antibodies and Cancer Therapy, Alan R. Liss, Inc., pp. 77-96). Alternatively, the monoclonal antibody can be produced using recombinant DNA methods (see, U.S. Pat. No. 4,816,567) or isolated from phage antibody libraries using the techniques described in Clackson et al (1991) Nature, 352:624-628; Marks et al (1991) J. Mol. Biol., 222:581-597.

The term “bispecific antibody” refers to an artificial antibody composed of two different monoclonal antibodies. They can be designed to bind either to two adjacent epitopes on CD38, thereby increasing both avidity and specificity, or bind two different antigens for numerous applications, but particularly for recruitment of cytotoxic T- and natural killer (NK) cells or retargeting of toxins, radionuclides or cytotoxic drugs for cancer treatment (Holliger & Hudson, Nature Biotechnology, 2005, 9, 23). The bispecific antibody may have a hybrid immunoglobulin heavy chain with a first binding specificity in one arm, and a hybrid immunoglobulin heavy chain-light chain pair (providing a second binding specificity) in the other arm. This asymmetric structure facilitates the separation of the desired bispecific compound from unwanted immunoglobulin chain combinations, as the presence of an immunoglobulin light chain in only one half of the bispecific molecule provides for a facile way of separation (WO 94/04690; Suresh et al., Methods in Enzymology, 1986, 121:210; Rodrigues et al., 1993, J. of Immunology 151:6954-6961; Carter et al., 1992, Bio/Technology 10:163-167; Carter et al., 1995, J. of Hematotherapy 4:463-470; Merchant et al., 1998, Nature Biotechnology 16:677-681.

Methods to prepare hybrid or bispecific antibodies are known in the art. In one method, bispecific antibodies can be produced by fusion of two hybridomas into a single ‘quadroma’ by chemical cross-linking or genetic fusion of two different Fab or scFv modules (Holliger & Hudson, Nature Biotechnology, 2005, 9, 23).

The term “chimeric antibody” refers to an antibody in which different portions are derived from different animal species. For example, a chimeric antibody may derive the variable region from a mouse and the constant region from a human. In contrast, a ‘humanised antibody’ comes predominantly from a human, even though it contains non-human portions. Specifically, humanised antibodies are human immunoglobulins (recipient antibody) in which residues from a hypervariable region of the recipient are replaced by residues from hypervariable regions of a non-human species (donor antibody) such as mouse, rat, rabbit or nonhuman primate having the desired specificity, affinity and capacity. In some instances, framework region (FR) residues of the human immunoglobulin are replaced by corresponding non-human residues. Furthermore, humanised antibodies may comprise residues that are not found in the recipient antibody or in the donor antibody. These modifications are made to further refine antibody performance. In general, the humanised antibody will comprise substantially all of at least one, and typically two, variable domains, in which all or substantially all of the hypervariable loops correspond to those of a non-human immunoglobulin and all or substantially all of the FRs are those of a human immunoglobulin sequence. The humanised antibody optionally also will comprise at least a portion of an immunoglobulin constant region (Fc), typically that of a human immunoglobulin.

Recombinant antibodies such as chimeric and humanised monoclonal antibodies can be produced by recombinant DNA techniques known in the art. Completely human antibodies can be produced using transgenic mice that are incapable of expressing endogenous immunoglobulin heavy and light chains genes, but which can express human heavy and light chain genes. The transgenic mice are immunized in the normal fashion with a selected antigen. Monoclonal antibodies directed against the antigen can be obtained using conventional hybridoma technology. The human immunoglobulin transgenes harboured by the transgenic mice rearrange during B cell differentiation, and subsequently undergo class switching and somatic mutation. Thus, using such a technique, it is possible to produce therapeutically useful IgG, IgA, IgM and IgE antibodies. For an overview of this technology for producing human antibodies, see Lonberg and Huszar (1995, Int. Rev. Immunol. 13:65-93). For a detailed discussion of this technology for producing human antibodies and human monoclonal antibodies and protocols for producing such antibodies. See, e.g., U.S. Pat. Nos. 5,625,126; 5,633,425; 5,569,825; 5,661,016; 5,545,806; each of which is incorporated herein by reference in its entirety. Other human antibodies can be obtained commercially from, for example, Abgenix, Inc. (Freemont, CA) and Genpharm (San Jose, CA).

The term “antibody” may also include a fusion protein of an antibody, or a functionally active fragment thereof, for example in which the antibody is fused via a covalent bond (e.g., a peptide bond), at either the N-terminus or the C-terminus to an amino acid sequence of another protein (or portion thereof, such as at least 10, 20 or 50 amino acid portion of the protein) that is not the antibody. The antibody or fragment thereof may be covalently linked to the other protein at the N-terminus of the constant domain.

In accordance with the present invention, the one or more anti-CD38 antibodies may be monoclonal antibodies.

In one embodiment, the anti-CD38 antibody binds to CD38 with a binding affinity of at least 100 nM, or at least 50 nM; and in a certain embodiment, at least 20 nM as determined using Biocore analysis.

As used herein, a “variant” may have at least 70%, 75%, 80%, 85%, 90%, 95%, 96%, 97%, 98%, or 99% overall sequence identity to the non-variant sequence.

In a certain embodiment, the one or more ant-CD38 antibodies may be selected from daratumumab, isatuximab, or combinations thereof. Those skilled in the art will appreciate that the one or more anti-CD38 antibodies for use in accordance with the present invention may comprise one or more additional agents. For example daratumumab may be provided as daratumumab and hyaluronidase (Daralex Faspro™). In one embodiment, two or more anti-CD38 antibodies may be used, where the antibodies bind to different epitopes on CD38. For example, daratumumab and isatuximab are known to bind to non-overlapping B-cell epitopes on CD38 with the result that the combination of daratumumab and isatuximab can act synergistically. In another embodiment, two or more CD38 antibodies can be used that bind to different epitopes on CD38.

The therapeutically effective amount of tinostamustine or a pharmaceutically acceptable salt, and of one or more anti-CD38 antibodies are administered to the patient in an amount which confers a therapeutic effect in accordance with the present invention on the treated subject, at a reasonable benefit/risk ratio applicable to any medical treatment. The therapeutic effect may be objective (i.e. measurable by some test or marker) or subjective (i.e. subject gives an indication of or feels an effect).

In certain embodiments of the invention, the amount of tinostamustine or pharmaceutically acceptable salt thereof administered to a patient in need thereof, and the amount of one or more anti-CD38 antibodies, has a molar ratio of 1:1000 to 1000:1, 1:700 to 700:1, 1:500 to 500:1, 1:300 to 300:1, 1:100 to 100:1, or 1:50 to 50:1.

### 3. Dosing and Formulations

A therapeutically effective amount of tinostamustine or a pharmaceutically acceptable salt thereof according to the present invention is believed to be one wherein tinostamustine or a pharmaceutically acceptable salt thereof is provided at a dosage range of from 0.3 mg/m² to 300 mg/m² body surface area of the patient or from 40 mg/m² to 150 mg/m² body surface area of the patient. In certain embodiments, tinostamustine or a pharmaceutically acceptable salt thereof may be provided at a dosage range from 50 to 100 mg/m² body surface area of the patient.

In other embodiments, tinostamustine or a pharmaceutically acceptable salt thereof is provided at a dosage range from 50 mg/m² to 80 mg/m² body surface area of the patient, for example from 55 to 70 mg/m², or from 55 to 65 mg/m² (e.g. 60 mg/m²).

The specific therapeutically effective dose level for any particular patient will depend upon a variety of factors including the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or contemporaneously with the specific compound employed; and like factors well known in the medical arts.

### 4. Administration

Suitable examples of the administration form of tinostamustine or a pharmaceutically acceptable salt thereof, or one or more anti-CD38 antibodies include without limitation oral, topical, parenteral, sublingual, rectal, vaginal, ocular, and intranasal. Parenteral administration includes subcutaneous injections, intravenous, intramuscular, intrasternal injection or infusion techniques.

In certain embodiments, tinostamustine or a pharmaceutically acceptable salt thereof, is administered parenterally. In certain embodiments, tinostamustine or a pharmaceutically acceptable salt thereof, is administered intravenously. In one embodiment, the one or more anti-CD38 antibodies (such as daratumumab), is administered parenterally. In certain embodiments, the one or more anti-CD38 antibodies (such as daratumumab), is administered intravenously.

In accordance with the present invention, tinostamustine or a pharmaceutically acceptable salt thereof may be administered intravenously to the patient in need thereof at a dosage level to the patient in need thereof of from 0.3 mg/m² to 300 mg/m² body surface area of the patient.

In embodiments, tinostamustine or a pharmaceutically acceptable salt thereof may be administered intravenously to the patient in need thereof at a dosage level to the patient in need thereof of from 40 mg/m² to 150 mg/m² body surface area of the patient. In certain embodiments, tinostamustine or a pharmaceutically acceptable salt thereof may be administered intravenously to the patient in need thereof at a dosage level to the patient in need thereof of from 50 mg/m² to 100 mg/m² body surface area of the patient.

In accordance with the present invention, tinostamustine or a pharmaceutically acceptable salt thereof can be administered to a patient in need thereof on days 1, 8 and 15 of a 28 day treatment cycle or on days 1 and 15 of a 28 day treatment cycle.

In certain embodiments, tinostamustine or a pharmaceutically acceptable salt thereof is administered on days 1 and 15 of a 28 day treatment cycle.

In embodiments of the present invention, tinostamustine or a pharmaceutically acceptable salt thereof or medicament comprising the same can be administered to a patient in need thereof over an infusion time of 60 minutes; or an infusion time of 45 minutes; or an infusion time of 30 minutes.

In certain embodiments, tinostamustine or a pharmaceutically acceptable salt thereof is administered over an infusion time of 60 minutes.

In certain embodiments, tinostamustine or a pharmaceutically acceptable salt is administered to the patient in need thereof at a dosage level of from 50 mg/m² to 100 mg/m² body surface area of the patient, on days 1 and 15 of a 28 day treatment cycle, over an infusion time of 60 minutes.

Tinostamustine or a pharmaceutically acceptable salt thereof may be administered concurrently with the one or more anti-CD38 antibodies.

Tinostamustine or a pharmaceutically acceptable salt thereof may be administered separately or sequentially with the one or more anti-CD38 antibodies. In embodiments, tinostamustine or a pharmaceutically acceptable salt thereof is administered prior to the separate or sequential administration of the one or more anti-CD38 antibodies. In further embodiments, tinostamustine or a pharmaceutically acceptable salt thereof is administered after to the separate or sequential administration of the one or more anti-CD38 antibodies.

In embodiments, tinostamustine or a pharmaceutically acceptable salt thereof may be administered on the same day(s) as administering the one or more anti-CD38 antibodies. In embodiments, the one or more anti-CD38 antibodies may be administered on one or more subsequent days on which tinostamustine or a pharmaceutically acceptable salt thereof is administered. For instance, the one or more anti-CD38 antibodies may be administered on 1, 2, 3, 4, 5, 6, 7, 8, 9, 10 or more subsequent days. Put into other words, tinostamustine or a pharmaceutically acceptable salt thereof may be administered on one or more days preceding the day on which one or more anti-CD38 antibodies are administered. Alternatively, tinostamustine or a pharmaceutically acceptable salt thereof may be administered on one or more days following the day on which one or more anti-CD38 antibodies are administered.

In certain embodiments, tinostamustine or a pharmaceutically acceptable salt thereof is administered prior to the separate or sequential administration of the one or more anti-CD38 antibodies, wherein the period between administration of tinostamustine or a pharmaceutically acceptable salt thereof and the one or more anti-CD38 antibodies is at least 12 hours. Suitably the period between administrations may be at least 24 hours, optionally at least 36 hours, and optionally still at least 48 hours. Advantageously, as described herein, prior administration of tinostamustine or a pharmaceutically acceptable salt thereof can increase the therapeutic efficacy of anti-CD38 antibodies.

When intended for oral administration, tinostamustine or a pharmaceutically acceptable salt thereof or medicament comprising the same may be in solid or liquid form, where semi-solid, semi-liquid, suspension and gel forms are included within the forms considered herein as either solid or liquid.

### 5. Pharmaceutical Composition

Tinostamustine or a pharmaceutically acceptable salt thereof or medicament comprising the same can be prepared for administration using methodology well known in the pharmaceutical art. Examples of suitable pharmaceutical formulations and carriers are described in “Remington's Pharmaceutical Sciences” by E. W. Martin.

As a solid composition for oral administration, tinostamustine or a pharmaceutically acceptable salt thereof can be formulated into a powder, granule, compressed tablet, pill, capsule, chewing gum, wafer or the like form. Such a solid composition typically contains one or more inert diluents or carriers. Any inert excipient that is commonly used as a carrier or diluent may be used in compositions of the present invention, such as sugars, polyalcohols, soluble polymers, salts and lipids. Sugars and polyalcohols which may be employed include, without limitation, lactose, sucrose, mannitol, and sorbitol. Illustrative of the soluble polymers which may be employed are polyoxyethylene, poloxamers, polyvinylpyrrolidone, and dextran. Useful salts include, without limitation, sodium chloride, magnesium chloride, and calcium chloride. Lipids which may be employed include, without limitation, fatty acids, glycerol fatty acid esters, glycolipids, and phospholipids.

In addition, one or more of the following can be present: binders such as carboxymethylcellulose, ethyl cellulose, microcrystalline cellulose, or gelatin; excipients such as starch, lactose or dextrins, disintegrating agents such as alginic acid, sodium alginate, corn starch and the like; lubricants such as magnesium stearate; glidants such as colloidal silicon dioxide; sweetening agents such as sucrose or saccharin; a flavoring agent such as peppermint, methyl salicylate or orange flavoring; and a coloring agent.

When tinostamustine or a pharmaceutically acceptable salt thereof compositions is in the form of a capsule (e.g. a gelatin capsule), it can contain, in addition to materials of the above type, a liquid carrier such as polyethylene glycol, cyclodextrin or a fatty oil.

Tinostamustine or a pharmaceutically acceptable salt thereof compositions can be in the form of a liquid, e.g. an elixir, syrup, solution, emulsion or suspension. The liquid can be useful for oral administration or for delivery by injection. When intended for oral administration, tinostamustine or a pharmaceutically acceptable salt thereof compositions can comprise one or more of a sweetening agent, preservatives, dye/colorant and flavor enhancer. In tinostamustine or a pharmaceutically acceptable salt thereof compositions for administration by injection, one or more of a surfactant, preservative, wetting agent, dispersing agent, suspending agent, buffer, stabilizer and isotonic agent can also be included.

The route of administration of tinostamustine or the one or more anti-CD38 antibodies, in some embodiments, is parenteral administration including, but not limited to, intradermal, intramuscular, intraperitoneal, intravenous, subcutaneous, intranasal, epidural, intranasal, intracerebral, intraventricular, intrathecal, intravaginal or transdermal. A desired mode of administration is left to the discretion of the practitioner, and will depend in part upon the site of the medical condition (such as the site of cancer). In another embodiment, tinostamustine or a pharmaceutically acceptable salt thereof or the one or more anti-CD38 antibodies or medicament comprising the same is administered intravenously.

Liquid forms of tinostamustine or a pharmaceutically acceptable salt thereof or medicament comprising the same, may be solutions, suspensions or other like form, and can also include one or more of the following: sterile diluents such as water for injection, saline solution, for example, physiological saline, Ringer's solution, isotonic sodium chloride, fixed oils such as synthetic mono or digylcerides, polyethylene glycols, glycerin, or other solvents; antibacterial agents such as benzyl alcohol or methyl paraben; and agents for the adjustment of tonicity such as sodium chloride or dextrose. A parenteral combination or composition can be enclosed in an ampoule, a disposable syringe or a multiple-dose vial made of glass, plastic or other material. Physiological saline is used as an adjuvant in one embodiment.

Tinostamustine or a pharmaceutically acceptable salt thereof or the one or more anti-CD38 antibodies or medicament comprising the same can be administered by any convenient route, for example by infusion or bolus injection, by absorption through epithelial or mucocutaneous linings, and by bolus.

Examples of compositions comprising tinostamustine or a pharmaceutically acceptable salt thereof are disclosed in WO2013/040286.

The present invention further provides pharmaceutical compositions comprising a pharmaceutically acceptable carrier, tinostamustine or a pharmaceutically acceptable salt thereof, and one or more anti-CD38 antibodies.

The term “pharmaceutically acceptable carrier” refers to a diluent, adjuvant or excipient, with which the compounds according to the present invention are administered. Such pharmaceutical carriers can be liquids, such as water and oils including those of petroleum, animal, vegetable or synthetic origin, such as peanut oil, soybean oil, mineral oil, sesame oil and the like. The carriers can be saline, gum acacia, gelatin, starch paste, talc, keratin, colloidal silica, urea, disaccharides, and the like. In addition, auxiliary, stabilizing, thickening, lubricating and coloring agents can be used. In embodiments, when administered to an animal, the compounds, compositions and pharmaceutically acceptable carriers are sterile. Water can be used as a carrier when the compounds are administered intravenously. Saline solutions and aqueous dextrose and glycerol solutions can also be employed as liquid carriers, particularly for injectable solutions. Suitable pharmaceutical carriers also include excipients such as starch, glucose, lactose, sucrose, gelatin, malt, rice, flour, chalk, silica gel, sodium stearate, glycerol monostearate, talc, sodium chloride, dried skim milk, glycerol, propylene glycol, water, ethanol and the like. The present compositions, if desired, can also contain minor amounts of wetting or emulsifying agents, or pH buffering agents. The pharmaceutically acceptable carrier or vehicle can be particulate, so that the compositions are, for example, in tablet or powder form. The carrier(s) can be liquid, with the compositions being, for example, an oral syrup or injectable liquid. In addition, the carrier(s) can be gaseous, or liquid so as to provide an aerosol composition useful in, for example inhalatory administration. Powders may also be used for inhalation dosage forms.

The present pharmaceutical compositions can take the form of solutions, suspensions, emulsions, tablets, pills, pellets, capsules, capsules containing liquids, powders, sustained-release formulations, suppositories, emulsions, aerosols, sprays, suspensions, or any other form suitable for use. Other examples of suitable pharmaceutical carriers are described in “Remington's Pharmaceutical Sciences” by E. W. Martin.

The pharmaceutical compositions can be prepared using methodology well known in the pharmaceutical art. For example, a composition intended to be administered by injection can be prepared by combining tinostamustine and one or more anti-CD38 antibodies with water, or other physiologically suitable diluent, such as phosphate buffered saline, to form a solution. A surfactant can be added to facilitate the formation of a homogeneous solution or suspension.

Pharmaceutical compositions can be formulated so as to allow a compound to be bioavailable upon administration of the composition to an animal, for example, a human. Pharmaceutical compositions can take the form of one or more dosage units, where for example, a tablet can be a single dosage unit, and a container of a compound may contain the compound in liquid or in aerosol form and may hold a single or a plurality of dosage units.

Pharmaceutical compositions according to the invention may be formulated according to the chosen route of administration. Examples of the administration form include without limitation oral, topical, parenteral, sublingual, rectal, vaginal, ocular and intranasal. Parenteral administration includes subcutaneous injections, intravenous, intramuscular, intrasternal injection or infusion techniques. In certain embodiments, the pharmaceutical compositions according to the present invention are administered parenterally.

Pharmaceutical compositions may be prepared in accordance with the dosage forms of tinostamustine and/or one or more anti-CD38 antibodies as described above.

The pharmaceutical compositions comprise an effective amount of tinostamustine and one or more anti-CD38 antibodies (e.g., daratumumab) such that a suitable dosage will be obtained. The correct dosage will vary according to the particular formulation, the mode of application, and its particular site and host. Other factors like age, body weight, sex, diet, time of administration, rate of excretion, condition of the host, drug combinations, reaction sensitivities and severity of the disease should be taken into account.

The compounds used in accordance with the present invention, or the pharmaceutical composition may be lyophilized. The lyophilized composition may be provided in a vial, which contains a specified amount of the pharmaceutical composition.

### 6. Kit

In a further aspect of the invention, there is provided a kit comprising tinostamustine or a pharmaceutically acceptable salt thereof or medicament comprising the same; and one or more anti-CD38 antibodies. In certain embodiments, the kit is provided together with instructions.

The instructions may advise administering tinostamustine or a pharmaceutically acceptable salt thereof; and/or one or more anti-CD38 antibodies, according to variables such as the state of the haematological cancer being treated; the age, body weight, general health, sex and diet of the patient; the time of administration, route of administration, and rate of excretion of the specific compounds employed; the duration of the treatment; drugs used in combination or contemporaneously with the specific compounds employed; and like factors well known in the medical arts.

### 7. Method of Treatment or Use

Although the treatment of cancer with tinostamustine and one or more anti-CD38 antibodies is described herein, those skilled in the art will appreciate that the treatment may comprise use of one or more other compounds or therapies. The one or more other compounds or therapies may be administered concurrently, sequentially or separately. The one or more other compounds or therapies may be selected from glucocorticoids and proteasome inhibitors. For example, the one or more other compounds or therapies may be selected from lenalidomide, bortezomib, melphalan, prednisone, dexamethasone and combinations thereof.

In embodiments of the present invention, the patient in need of said treatment is given radiotherapy with (including prior to, during or after) treatment of the cancer with tinostamustine or a pharmaceutically acceptable salt thereof, and one or more anti-CD38 antibodies. In embodiments of the present invention, the patient is treated with tinostamustine or a pharmaceutically acceptable salt thereof, one or more anti-CD38 antibodies, and radiotherapy. In certain embodiments, the patient is given radiotherapy treatment prior to the treatment with tinostamustine or a pharmaceutically acceptable salt thereof and one or more anti-CD38 antibodies. The radiotherapy may be given at a dose of 1 to 5 Gy over 5-10 consecutive days, such as 2 Gy over 5-10 consecutive days.

To provide a more concise description, some of the quantitative expressions given herein are not qualified with the term “about”. It is understood that, whether the term “about” is used explicitly or not, every quantity given herein is meant to refer to the actual given value, and it is also meant to refer to the approximation to such given value that would reasonably be inferred based on the ordinary skill in the art, including equivalents and approximations due to the experimental and/or measurement conditions for such given value.

While the foregoing disclosure provides a general description of the subject matter encompassed within the scope of the present invention, including methods, as well as the best mode thereof, of making and using this invention, the following examples are provided to further enable those skilled in the art to practice this invention and to provide a complete written description thereof. However, those skilled in the art will appreciate that the specifics of these examples should not be read as limiting on the invention, the scope of which should be apprehended from the claims and equivalents thereof appended to this disclosure. Various further aspects and embodiments of the present invention will be apparent to those skilled in the art in view of the present disclosure.

### EXAMPLES

### Materials and Methods

EDO-S101 (tinostamustine) was obtained from Mundipharma (Basel, Switzerland).

Antibodies Daratumumab monoclonal antibody (mAb) was obtained from the Pharmacy Department of the University Hospital of Salamanca. Daratumumab isotype control IgG1 was purchased from Sigma-Aldrich (San Luis, MO, USA).

Mouse anti-human CD56-PE, CD45-PerCPcy5, CD38-APC and the IgG1 κ-APC isotype control were obtained from BD Biosciences (San Jose, CA, USA). Mouse anti-human MICA-Alexa Fluor 488, MICB-Alexa Fluor 488, ULBP1-PE, ULBP3-PE, CD155-APC (PVR), CD112-APC (Nectin-2) and the corresponding isotype controls IgG2B-Alexa Fluor 488, IgG2A-PE and IgG1 κ-APC were purchased from R&D Systems (Minneapolis, MN, USA). CD38me-FITC (Anti-human CD38 multi-epitope) was from obtained from Cytognos (Salamanca, Spain) and CD45-APC was obtained from Immunostep.

All primary antibodies used in western blot analysis (anti-CD38, anti-c-Jun, anti-c-Fos, anti-IRF1, anti-NFκB and anti-α-tubulin) were obtained from Cell Signalling (Boston, MA, USA). Horseradish peroxidase-conjugated secondary antibodies (anti-mouse or anti-rabbit) were from GE Healthcare (Chicago, IL, USA).

### Cells

Human myeloma cell lines—MM1S, and U266 were purchased from ATCC (Manassas, VA, USA). RPMI-8226, KMS12-BM, JJN3 and MOLP-8 cell lines were obtained from DSMZ (Braunschweig, Germany). BM samples from MM patients were obtained following approval from the University Hospital of Salamanca Ethical Committee (CEIm ref.: 2021/06/799) and written informed consent from patients. Research with human samples was conducted in accordance with the Declaration of Helsinki guidelines.

NK Cells—Mononuclear cells were isolated from buffy coats from healthy donors using density gradient centrifugation with Ficoll. NK cells were isolated from mononuclear cells in an autoMACS® Pro Separator (Miltenyi Biotec, Bergisch Gladbach, Germany).

Monocytes—Mononuclear cells were isolated from buffy coats from healthy donors using density gradient centrifugation with Ficoll. CD14+ monocytes were magnetically isolated from mononuclear cells in an autoMACS® Pro Separator, and said monocytes were cultured for 7 days in the presence of GM-CSF (10 ng/ml) to induce differentiation to macrophages.

Cell Culture Cell culture media, serum, and penicillin-streptomycin were purchased from Invitrogen Corporation (Gaithersburg, MD, USA). Cell lines were cultured in RPMI-1640 medium supplemented with 2 mM L-glutamine, 100 U/mL penicillin, 100 mg/mL streptomycin and 10% fetal bovine serum (FBS) at 37° C. and 5% CO₂. MOLP-8, as an exception, was cultured with 20% FBS.

Cell Treatment with EDO-S101 MM cells (MM1S, JJN3, KMS12-BM, RPMI-8226, U266, MOLP-8) were either treated with a control (0 μM EDO-S101, DMSO vehicle) or treated with EDO-S101 (0.1, 0.5, 1 or 2.5 μM in DMSO) for 48 hours, unless indicated otherwise.

Flow cytometry Mouse anti-human CD138-FITC, CD56-PE, CD45-PerCP-Cy5.5, CD38-APC and the IgG1 κ-APC isotype control were obtained from BD Biosciences (San Jose, CA, USA). Mouse anti-human MICA-Alexa Fluor 488, MICB-Alexa Fluor 488, ULBP1-PE, ULBP3-PE, CD155-APC (PVR), CD112-APC (Nectin-2) and the corresponding isotype controls IgG2B-Alexa Fluor 488 and IgG2A-PE were purchased from R&D Systems (Minneapolis, MN, USA). Mouse anti-human ULBP-2 and anti-mouse-Alexa Fluor 488 were obtained from Sigma-Aldrich. CD38me-FITC (anti-human CD38 multi-epitope) was acquired from Cytognos (Salamanca, Spain) and CD45-APC from Immunostep (Salamanca, Spain).

For surface staining, cells were incubated with the appropriate antibodies and 7AAD (Immunostep) for 15 min in the dark. Data were acquired using a FACSCalibur cytometer (BD Biosciences) and analyzed using Infinicyt™ software (Cytognos). Specifically, the expression of CD38 or ligands for NK cell activating receptors (MICA, MICB, ULBP1-3, PVR and CD112) in MM cell lines was determined on the viable cell population (7AAD-). In primary myeloma cells, the expression of CD38 was analyzed over the viable myeloma population using the panel CD138-FITC/CD56-PE/7AAD/CD38-APC, whereas MICA/B-AF488/CD56-PE/7AAD/CD38-APC panel was used to determine MICA/B expression. Normalized median fluorescence intensity (MFI) was calculated by dividing median fluorescence obtained with the specific antibody by median fluorescence of the corresponding isotype control. MFI for each treatment was expressed as a percentage relative to the control (100%, cells cultured in media with DMSO).

Western Blot Analysis Cells were collected and centrifuged at 1200 rpm for 5 minutes at 4° C. Cells were washed with PBS and lysed in ice-cold lysis buffer [140 mmol/L NaCl, 10 mmol/L EDTA, 10% glycerol, 1% NP40, 20 mmol/L Tris (pH 7), 1 μMol/L pepstatin, 1 μg/mL aprotinin, 1 μg/mL leupeptin, 1 mmol/L sodium orthovanadate]. Samples were centrifuged at 10,000×g at 4° C. for 10 minutes, and supernatants were transferred to new tubes and western blots were performed following standard procedures. CD38 and anti-α-tubulin primary antibodies used in Western blot analyses were obtained from Cell Signaling (Boston, MA, USA). The horseradish peroxidase-conjugated secondary antibodies were from GE Healthcare (Chicago, IL, USA).

Immunofluorescence After appropriate treatments in vitro, cells were placed on glass slides coated with poly-L-lysine followed by 15 minutes incubation in a cell culture incubator. Cells were fixed with 4% paraformaldehyde for 30 minutes, blocked with BSA 100 μg/mL for 1 h at room temperature and incubated with a primary antibody (anti-CD38 antibody (1:100)) overnight at 4° C. The incubation with the anti-mouse secondary antibody, labelled with Alexa Fluor 488, was performed in darkness for 1 h at room temperature. After three washes for 5 min in PBS, nuclei were stained with DAPI (Sigma) 1:1000 in PBS for 5 min at room temperature. Finally, cells were washed three times for 10 min in PBS and slides were then mounted using VECTASHIELD Mounting Medium (Vector Laboratories, Burlingame, CA). Fluorescent images were acquired with a LEICA SP5 DMI-6000B confocal microscope (Leica, Wetzlar, Germany) with a 63.0× lens zoomed in 6×.

Real time quantitative PCR (RT-qPCR) Total RNA was isolated using the RNeasy Mini Kit (Qiagen, Hilden, Germany). Following reverse transcription, PCR was performed using TaqMan Gene Expression Assays (Applied Biosystems, Foster City, CA, USA): CD38 (Hs01120071_m1), MICA (Hs00741286_m1), MICB (Hs00792952_m1) and GAPDH (Hs99999905_m1). The results were expressed as fold change relative to expression of the target gene in control cells, using endogenous expression of GAPDH for normalization.

Chromatin Immunoprecipitation RPMI-8226 and MM.1S cell lines were treated with DMSO or tinostamustine (2.5 μM) for 48 hours. Then, the cells were fixed with Pierce™ fresh methanol-free formaldehyde (ThermoFisher) for 15 minutes and prepared for sonication with the truChIP Chromatin Shearing Kit (Covaris, Woburn, MA, USA), following the manufacturer's instructions and as previously described (Garcia-Gomez et al., Nat Commun 12: 421, 2021).

For ChIP-qPCR, samples were diluted 1/10, and 4 μL and specific primers (Supplementary Table 1) were used for each reaction. RT-qPCR was performed in technical triplicates for each biological replicate, using LightCycler® 480 SYBR Green Mix (Roche). The relative amount of immunoprecipitated DNA was compared to the input DNA for each condition.

In vivo models—Murine experiments were conducted according to University of Salamanca guidelines for the use of laboratory animals, and after granted permission from the University of Salamanca Animal Ethical Committee for animal experimentation and the Agriculture and Livestock Council of Junta de Castilla y León (Registry Number 0000061; Registered User Center: ES372740000046).

MM1S-luc cells (3×10⁶) were injected intravenously (i.v.) into CB17-SCID mice which were determined to have normal NK cells, monocytes and granulocytes (The Jackson Laboratory, Bar Harbor, ME, USA). Tumor development was monitored by non-invasive bioluminescence imaging (BLI) with a Xenogen IVIS 50 system (Caliper Life Sciences, Hopkinton, MA, USA). Mice were randomized into four groups (n=5/group): one receiving vehicle (i.v., weekly), another group receiving EDO-S101 (15 mg/kg i.v., weekly on Tuesdays), another group receiving daratumumab (8 mg/kg i.p., weekly on Wednesdays) and the last group receiving the double combination of EDO-S101+daratumumab. Both drugs were administered over a period of 4 weeks.

For the first subcutaneous human plasmacytoma model, NSG mice (Charles River Laboratories, Wilmington, MA, USA) were subcutaneously inoculated into the right flank with 3×10⁶ MM1S in 100 μL RPMI 1640 medium and 100 μL of Matrigel (BD Biosciences). When tumors developed, mice were injected with human NK cells isolated from buffy coats from healthy donors and randomized (4 mice/group) to receive: EDO-S101 (30 mg/kg, iv weekly), daratumumab (8 mg/kg, ip weekly), a combination thereof, or vehicle (PBS vehicle solution with 15% HPBCD, 1.5% acetic acid, and 1.25% NaHCO₃). Both, NK cells and the treatments were administered over four consecutive weeks. Caliper measurements of the tumor diameters were performed three times a week, and the tumor volume was estimated as the volume of a 3D ellipse using the following formula: V=4/3π×(a/2)×(b/2)², where “a” and “b” correspond to the longest and shortest diameter, respectively. Animals were killed when their tumors reached 2 cm. Differences in tumor volumes between control and treated groups were evaluated using one-way analysis of variance and Dunn's post-hoc tests.

For the second subcutaneous human plasmacytoma model, CB17-SCID mice (The Jackson Laboratory) were used. CB17-SCID mice were randomised into groups (n=5). The design of the experiment (treatment doses and time schedule) was identical to the experiment described above with NSG mice except for the inoculation of NK cells, since CB17-SCID mice have their own functional NK cells.

Evaluation of daratumumab binding to myeloma cells To evaluate daratumumab binding to myeloma cells, MM cell lines were first pre-treated with tinostamustine (1 or 2.5 μM) or DMSO for 48 hours. Then cells were washed to remove tinostamustine or DMSO from the culture medium, and daratumumab (10 μg/ml) was added and incubated for 30 minutes. After that time, cells were washed with PBS twice and incubated during 30 additional minutes with anti-human-IgG1-AlexaFluor488 (Invitrogen, Carlsbad, CA, USA). Cells were acquired in a FACSCalibur cytometer and normalized MFI was calculated as described above.

ADCC, CDC and apoptosis via cross-linking To assess the effect of tinostamustine pre-treatment on cytotoxic mechanisms mediated by daratumumab, MM cell lines were first incubated in presence of tinostamustine (2.5 μM) or DMSO for 48 hours. Cells were then washed to remove tinostamustine or DMSO from the culture medium, and the corresponding cytotoxic assay (ADCC, CDC or apoptosis via cross-linking) was performed.

For the evaluation of ADCC, tinostamustine or DMSO pre-treated cells were cocultured with NK cells obtained from healthy donors in the presence of daratumumab (1 μg/ml) or the corresponding isotype control for 4 hours (ratio 1:1). Briefly, peripheral blood mononuclear cells (PBMCs) were isolated from healthy donor buffy coats by Lymphoprep density gradient centrifugation. Afterwards, NK cells were isolated from PBMNCs using NK Cell Isolation Kit (Miltenyi Biotec, Bergisch Gladbach, Germany) in an autoMACS® Pro Separator (Miltenyi Biotec) following manufacturer's instructions. For CDC analysis, pre-treated MM cells were cultured for 4 hours in the presence of daratumumab (1 μg/mL) or the corresponding isotype control plus 10% human serum as a complement source. To evaluate apoptosis via cross-linking, pre-treated MM cells were incubated during 24 hours with daratumumab (1 μg/mL) or the corresponding isotype control plus F(ab)₂ fragments (10 μg/mL) for the induction of cross-linking. In all three assays, the cytotoxic effect on MM cells was evaluated by flow cytometry with Annexin V/7AAD staining after treatment with daratumumab. Specifically for ADCC, additional staining with CD38me and CD45 was included to identify MM cells (CD38+CD45−) in the NK cells/myeloma coculture.

Ex vivo analysis of apoptosis in BM samples from myeloma patients BM samples from myeloma patients were lysed to remove red blood cells and cultured as previously described (Ocio et al., Blood 113: 3781-3791, 2009), in the presence of daratumumab (10 μg/mL), tinostamustine (0.5-1 μM), or the double combination of daratumumab+tinostamustine or daratumumab's isotype control for 24 hours. After the incubation period, cells were collected in BD Trucount™ Absolute Counting Tubes (Becton Dickinson, Franklin Lakes, NJ) and stained with the corresponding antibodies (CD38me-FITC/CD45-PerCP-Cy5.5/CD56-PE) for flow cytometry analysis. The percentage of eliminated cells was calculated following manufacturer's instructions on myelomatous plasma cells (CD38+bright, CD45−/low, SSClow/intermediate, CD56−/+) and normal lymphocytes (CD45++, SSClow) as described elsewhere (Verkleij et al., Cancers (Basel) 12:E3713, 2000).

In vivo evaluation of the efficacy of the combination of tinostamustine+daratumumab in subcutaneous plasmacytoma models For the first subcutaneous human plasmacytoma model, NOD.Cg-Prkdcscidll2rgtm1Wji/SzJ (NSG) mice (16 female mice, 5-6 weeks old; from Servicio de Experimentación Animal O.M.G., USAL, Salamanca, Spain) were subcutaneously injected with 3×10⁶ MM.1S cells in 100 μL RPMI-1640 medium+100 μL Matrigel (BD Biosciences) into the right flank. When tumors became palpable, mice were randomized into 4 groups (4 mice/group) to receive: tinostamustine (30 mg/kg, i.v., weekly), daratumumab (8 mg/kg, i.p., weekly), the double combination or vehicle [PBS vehicle solution with 0.4 mg/kg daratumumab isotype control IgG1, 15% 2-hydroxypropyl-ß-cyclodextrin (HPBCD), 1.5% acetic acid and 1.25% NaHCO₃]. Tinostamustine was administered 24 hours before daratumumab as appropriate. In addition, all mice were i.v. injected with human NK cells isolated from healthy donor buffy coats as previously described (1.4-2×10⁶/mouse/weekly, depending on NK cells availability in each buffy coat) coinciding with daratumumab regimen. Both, human NK cells and the treatments were administered during 4 consecutive weeks. Caliper measurements of tumor diameters were performed three times a week, and the tumor volume was estimated as the volume of a 3D ellipse using the following formula: V=4/3π×(a/2)×(b/2)², where “a” and “b” correspond to the longest and shortest diameter, respectively. Animals were sacrificed when tumors reached 2 cm in longest diameter or if distress signs were observed. Statistical differences in tumor volumes between the different groups were evaluated using one-way analysis of variance (ANOVA) and Tukey's HSD post-hoc tests.

For the second subcutaneous human plasmacytoma model, CB17-SCID mice (20 female mice, 5-6 weeks old; from The Jackson Laboratory) were used. The design of the experiment (treatment doses and time schedule) was identical to the one performed with NSG mice except for the inoculation of human NK cells, since CB17-SCID mice hold their own functional NK cells (Dorshkind et al., J Immunol 134:3798-3801, 1985).

Animal experiments were conducted according to institutional guidelines for the use of laboratory animals, and after granted permission from the University of Salamanca Animal Ethical Committee for animal experimentation and the Agriculture and Livestock Council of Junta de Castilla y León (Registry Numbers: 0000061 and 292; Registered User Center: ES372740000046).

Statistical analyses Statistical analyses were carried out using GraphPad Prism software v6 (GraphPad Software), as indicated for each experiment. Otherwise specified, data are summarized as the mean±SEM. Student's t test or ANOVA tests were used to determine statistical significance. P-values lower than 0.05 were considered statistically significant.

### Example 1—EDO-S101 Increases CD38 Expression in MM Cell Lines

The effect of tinostamustine on CD38 expression was evaluated by flow cytometry in seven MM cell lines with different basal expression of this protein.

Cytometry, western blot analysis, immunofluorescence and qPCR was performed in accordance with the methods outlined above to analyse the expression of CD38 in MM cell lines treated with EDO-S101. The indicated MM cells were treated with EDO-S101 at the indicated concentrations in accordance with the general methods outlined above, unless indicated otherwise.

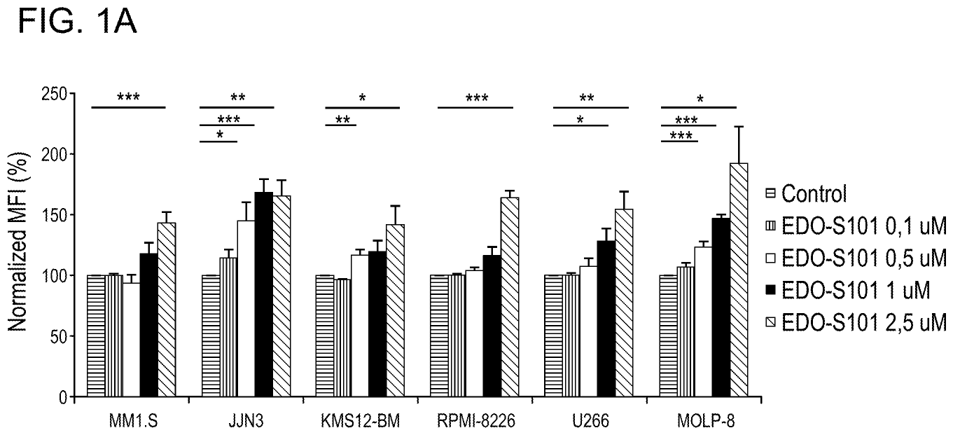

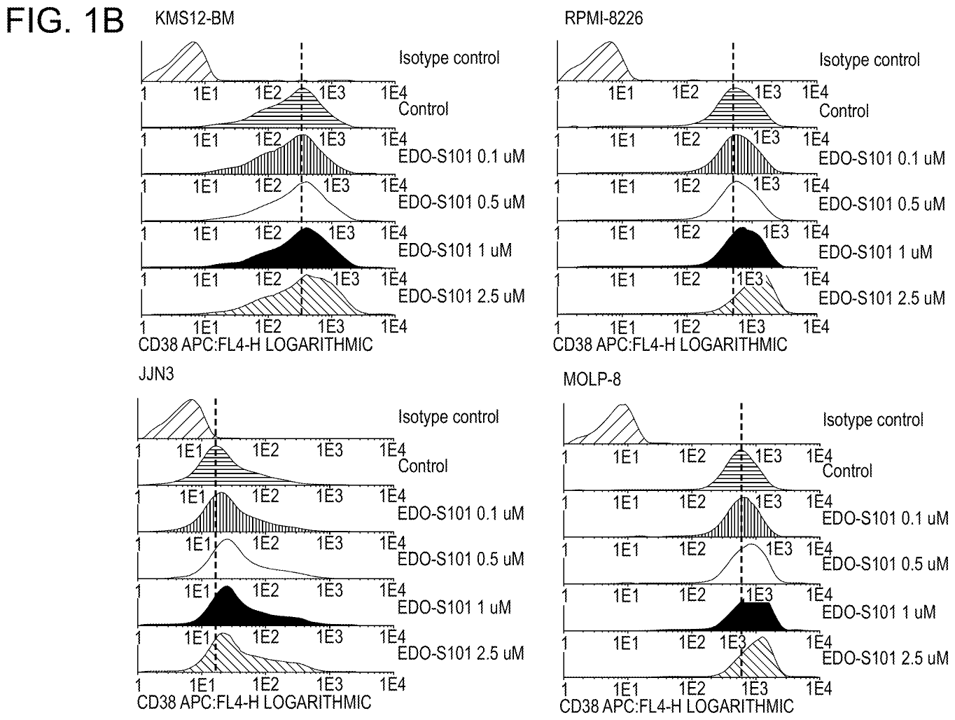

Treatment of MM cells (MM1.S, JJN3, KMS12-BM, RPMI-8226, U226, MOLP-8) with EDO-S101 (0.1, 0.5, 1 and 2.5 μM) for 48 hours significantly increased CD38 surface expression in all cell lines investigated relative to a control (0 μM EDO-S101, DMSO), as determined by flow cytometry (FIG. 1A; each bar shows mean±SEM (n=3); *p<0.05, **p<0.01, ***p<0.001 by the Student t test), with the only exception being NCI-H929 cells (data not shown).

As also shown in FIG. 1B, treatment with EDO-S101 (0.1, 0.5, 1 and 2.5 μM) significantly increased surface CD38 expression in the MM cell lines (KMS12-BM, RMPI-8226, JJN3, MOLP-8) compared to the control (0 μM EDO-S101; FIG. 1B). The vertical dashed line represents the modal fluorescence for control cell populations. Cells treated with EDO-S101 show an increased modal fluorescence, as evidenced by a peak to the right of the dashed line. These data therefore affirm increased CD38 surface expression in MM cells treated with EDO-S101.

It should be noted that these effects were observed both in cell lines with low/medium basal levels of CD38 (e.g., U266, KMS12-BM and JJN3) and in those with high basal levels (e.g., RPMI-8226 and MOLP-8).

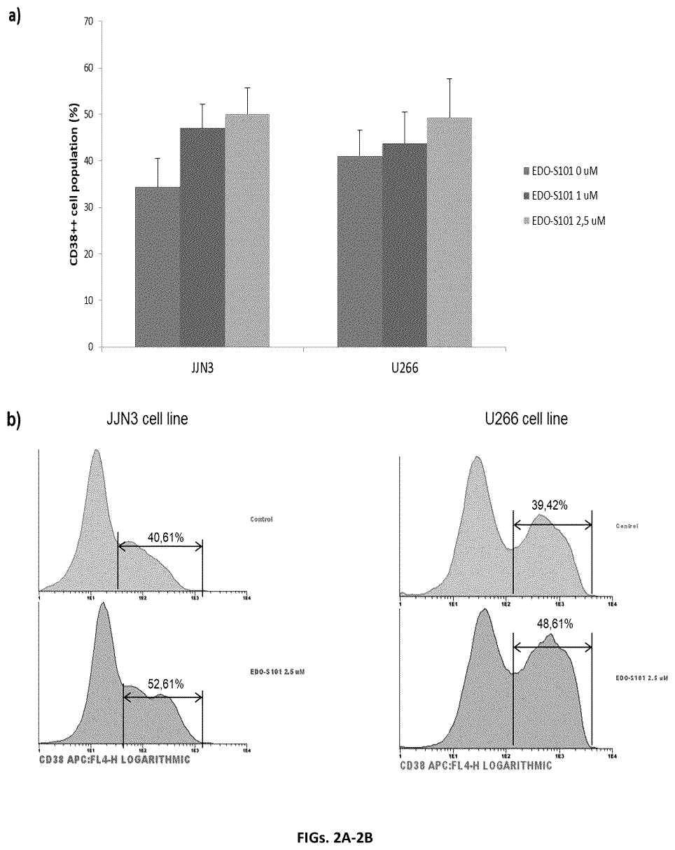

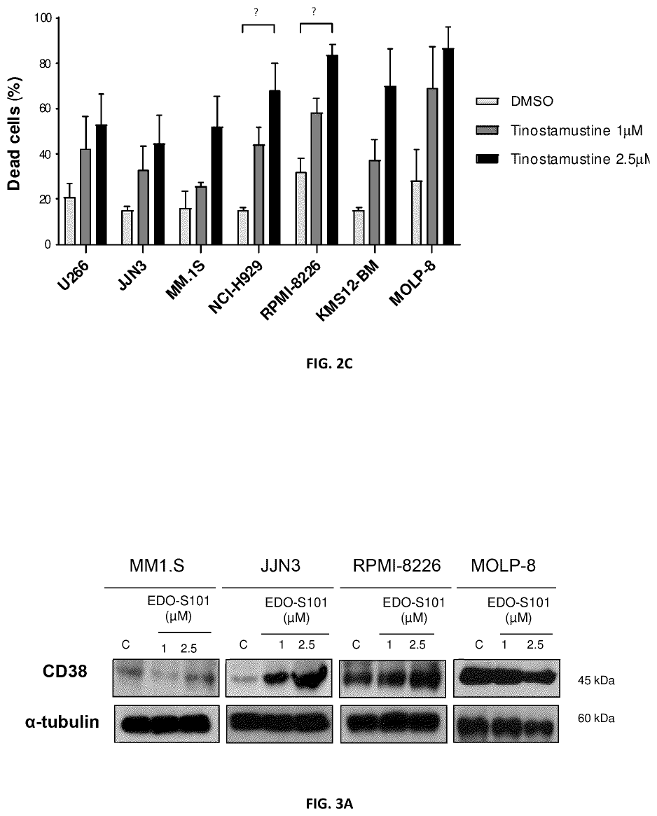

Moreover, in MM cell lines (e.g. JJN3, U266, and MM.1S) that are heterogeneous for CD38 surface expression, with a CD38⁺ and a CD38^{-/low} population (e.g., JJN3 and MM.1S), treatment with EDO-S101 (1 and 2.5 μM) was found to increase the percentage of the CD38⁺ cell population, as shown in FIG. 2A, each bar shows mean±SEM (n=3)). At least the increase in JJN3 cells were statistically significant, with the change in MM. 1S (data not shown) (and a 3rd line U266) trending up. The histograms provided in FIG. 2B indicate the percentage of CD38+ cells in the population of control and EDO-S101 treated MM cells, as determined by integration of the histogram. Treatment with tinostamustine significantly increased the percentage of cells in the first population in the JJN3 cell line, and a similar tendency was observed for U266 cell line (FIG. 2B) and MM.1S cell line (data not shown).

Note that the percentage of viable cells after treatment with the highest dose of tinostamustine ranged between 25.6% for the most sensitive cell line (MOLP-8) to 55.48% for the less sensitive one (JJN3) (FIG. 2C). For subsequent experiments MM.1S, JJN3, RPMI-8226 and MOLP-8 cell lines were chosen due to their different CD38 basal expression and their sensitivity to tinostamustine.

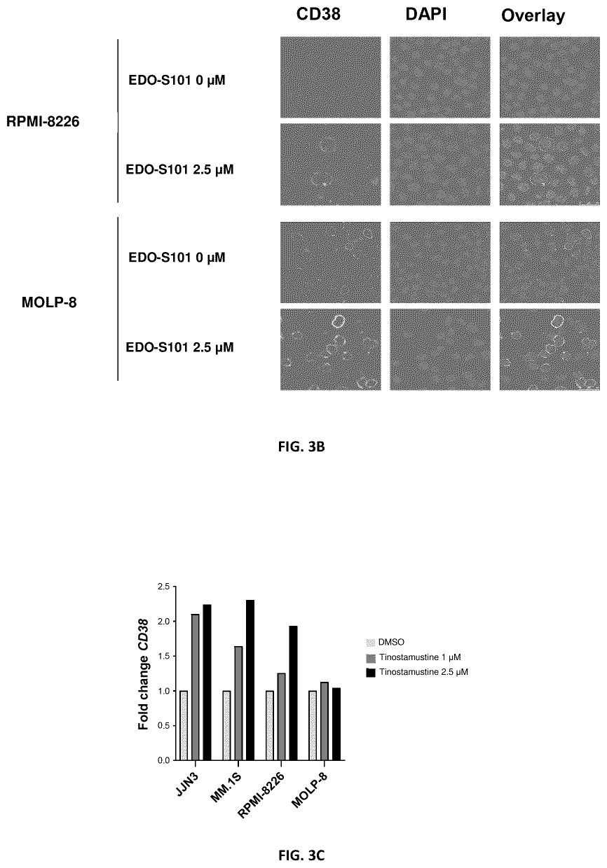

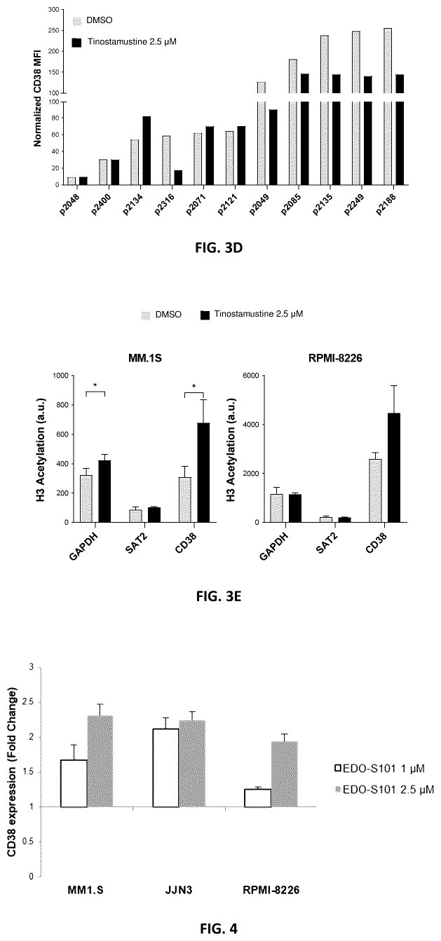

Further western blot analysis (FIG. 3A) confirmed that treatment of MM cell lines (MM1.S, JJN3, RPMI-8226) with EDO-S101 (1 and 2.5 μM) increased CD38 surface expression. Immunofluorescence (FIG. 3B) provided further confirmation that treatment of MM cell lines (RPMI-8226, MOLP-8) with EDO-S101 (2.5 μM) increased CD38 expression, relative to control cells (0 μM EDO-S101; DMSO). Increased fluorescence signal was observed for CD38 in MM cells treated with EDO-S101. DAPI is provided as a marker for nuclear DNA.

The expression of CD38 at a transcriptional level following treatment with EDO-S101 (1 and 2.5 μM) for 36 hours was further analysed using qPCR in the MM cell lines: MM1.S, JJN3, and RPMI-8226. As shown in FIG. 4, CD38 expression at the transcriptional level was increased by EDO-S101 in the MM cell lines tested.

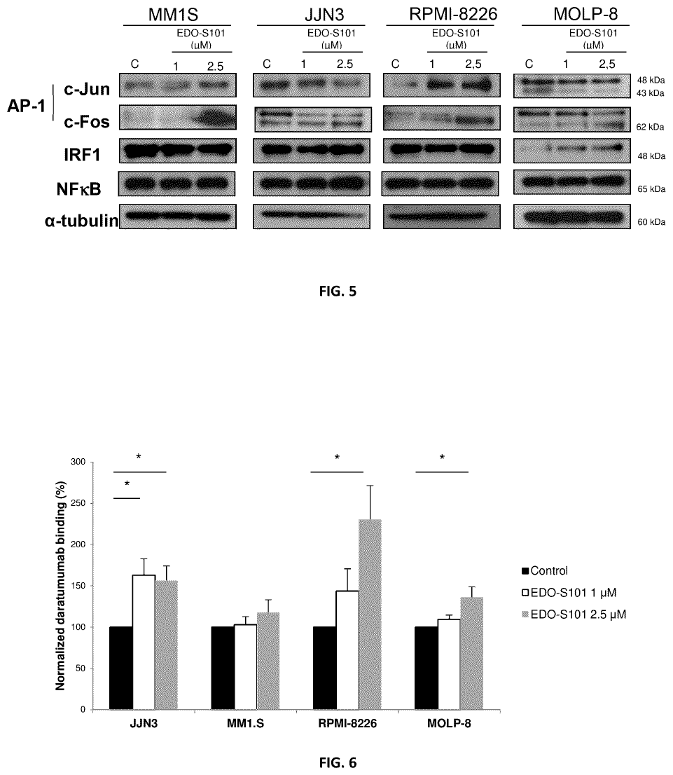

To determine the mechanism by which EDO-S101 increases CD38 expression, the expression of the transcription factors NF-κB, AP-1 and IRF-1 which are known to be involved in regulating the expression of CD38 was evaluated. Treatment with EDO-S101 (1 and 2.5 μM) for 48 h increased the expression of c-Jun and c-Fos (subunits of the transcription factor AP-1) in two (MM1.S and RPMI-8226) of the four myeloma cell lines tested (FIG. 5). Additionally, EDO-S101 increased IRF-1 expression in the MOLP-8 cell line (FIG. 5).

Although the increase in CD38 surface expression observed by flow cytometry was confirmed by Western blot and RT-qPCR in MM.1S, JJN3 and RPMI-8226 cell lines, these results were not observed in MOLP-8 cells, suggesting a different tinostamustine mediated mechanism of increased surface expression of CD38 in this line (FIGS. 3A and 3C).

CD38 expression was further examined by flow cytometry in malignant plasma cells from ex vivo cultures of 11 MM patients' bone marrow samples treated with tinostamustine (2.5 μM) for 48 hours. As shown in FIG. 3D, tinostamustine (2.5 μM) slightly increased CD38 expression in primary myeloma cells in 3 out of 11 patients analyzed (p2134, p2071 and p2121), corresponding to some of those with low basal antigen expression, but this effect was not observed in any of the patients with high CD38 basal expression.

Consistent with the fact that tinostamustine has alkylating histone deacetylase inhibitor activity, levels in histone acetylation were found to be associated with the increased CD38 expression observed in MM cell lines. Specifically, the acetylation status of histone H3 was determined using a ChIP-qPCR analysis of CD38 in MM.1S and RPMI-8226 cell lines after treatment with DMSO or tinostamustine (2.5 μM). In this sense, CD38 histone H3 acetylation levels were elevated after treatment with tinostamustine in comparison to DMSO-treated cells in MM.1S cell line, and similar results were obtained with RPMI-8226 cells (FIG. 3E).

These in vitro data indicate treatment of MM cell lines with EDO-S101 increases the surface expression of CD38 as well as levels of CD38 transcription, relative to control MM cells that were not treated with EDO-S101.

### Example 2—EDO-S101 Increases Binding of Daratumumab to MM Cell Lines

This example demonstrates that the increase in CD38 antigen expression promoted by tinostamustine was able to improve the binding of daratumumab (an anti-CD38 monoclonal antibody) to myeloma cells.

Flow cytometry was used to analyse the binding of daratumumab to MM cells pre-treated with EDO-S101. The anti-human-IgG1-Alexa Fluor 488 (Invitrogen, Carlsbad, CA, USA) antibody recognizes and binds daratumumab's Fc region and, therefore, it can be detected using flow cytometry.

MM cells (JJN3, MM1.S, RPMI-8226, MOLP-8) were treated with EDO-S101 (1 μM or 2.5 μM) in accordance with the general methods outlined above. Following treatment, the cells were washed with PBS, and incubated at 37° C. for 30 minutes with 10 μg/ml daratumumab. Cells were then washed with PBS, anti-human-IgG1-Alexa Fluor 488 was added, and the cells were incubated at 37° C. for a further 30 minutes. Flow cytometry was then performed in accordance with the methods outlined above to calculate normalised MFI expression of anti-human bound to daratumumab on the myeloma cells.

FIG. 6 (each bar shows mean±SEM (n=3); *p<0.05 by the Student t test) demonstrates that treatment of MM cells (JJN3, RPMI-8226 and MOLP-8 MM) with EDO-S101 increases the binding of daratumumab to MM cells, relative to control MM cells (0 μM EDO-S101).

These data show that EDO-S101-induced overexpression of CD38 significantly increased binding of daratumumab to multiple myeloma cells, therefore indicating increased efficacy of daratumumab against the multiple myeloma cells.

### Example 3—EDO-S101 Increases the Immunogenicity of MM Cell Lines

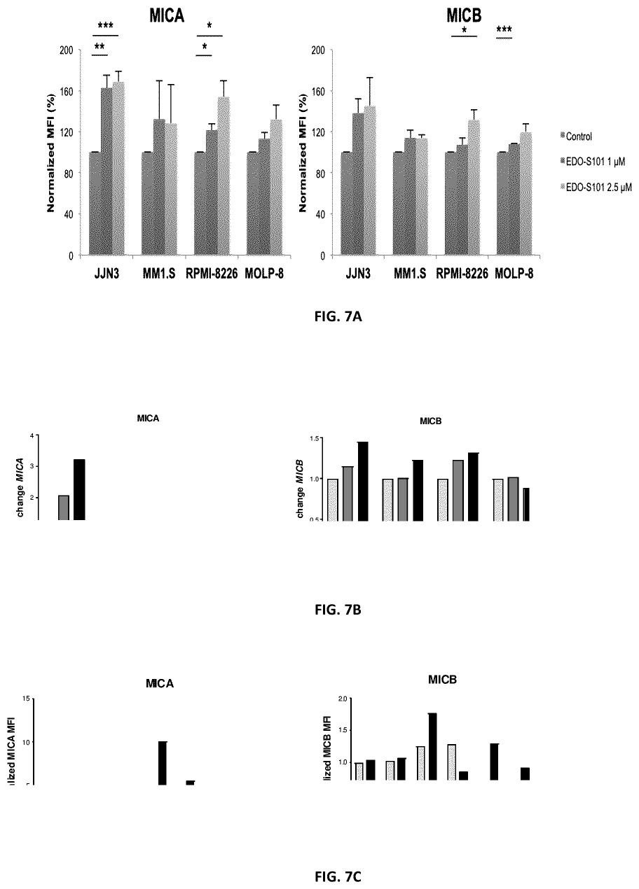

In this example, increased immunogenicity of MM cells following treatment with EDO-S101 was evaluated. Specifically, the data presented herein showed that tinostamustine increased the surface expression of the NKG2D ligand MICA in all cell lines tested, reaching statistical significance in JJN3 and RPMI-8226 cell lines. Similar results were obtained for MICB, another NKG2D ligand (FIG. 7A).

MM cells (JJN3, MM1.S, RPMI-8226, MOLP-8) were treated with EDO-S101 (1 and 2.5 μm) for 48 hours in accordance with the methods outlined above. After incubation, expression of several ligands for NK cell activating receptors: MICA, MICB, ULBP1, ULPB2, ULPB3, CD155 (PVR) and CD112 (Nectin-2) were measured using flow cytometry. Flow cytometry was then performed in accordance with the methods outlined above to calculate.

As shown in FIG. 7A and in Table 1 below, treatment with EDO-S101 for 48 hours increased the surface expression of MICA and MICB in all cell lines tested. These results provide a further scientific rationale for combining EDO-S101 and anti-CD38 antibodies. Increased expression of ligands for NK cell activating receptors leads to the direct activation of NK cells and thus a direct NK attack on the tumor cell. This mechanism complements that of the anti-CD38 antibodies, which in part act through NK-cell mediated ADCC to exert their cytotoxic effect.

TABLE 1
Normalized MFI of NK cell activating ligands analysed by flow cytometry in MM cell lines.

| | EDO-S101 | | | | | | |
|---|---|---|---|---|---|---|---|
| | (μM) | MICA | MICB | ULBP2 | ULBP3 | CD155 | CD112 |
| JJN3 | 0 | 100 | 100 | 100 | — | 100 | — |
| | 1 | 162.9 ± 11.8 | 137.6 ± 14 | 110.5 ± 9.5 | — | 161.4 ± 14.3 | — |
| | 2.5 | 168.8 ± 10.3 | 144.9 ± 28 | 112.9 ± 13.4 | — | 156.8 ± 24.7 | — |
| MM1.S | 0 | 100 | 100 | 100 | — | 100 | — |
| | 1 | 131.9 ± 37.6 | 113.8 ± 7.6 | 95.8 ± 4.5 | — | 113.3 ± 3.9 | — |
| | 2.5 | 128 ± 38.2 | 113.4 ± 3.3 | 96.3 ± 12.3 | — | 111.5 ± 15.3 | — |
| RPMI-8226 | 0 | 100 | 100 | — | 100 | 100 | 100 |
| | 1 | 121.3 ± 5.9 | 107.1 ± 6.6 | — | 111.7 ± 14.8 | 123.9 ± 6.1 | 116.6 ± 13.2 |
| | 2.5 | 153.6 ± 16.1 | 131.3 ± 9.4 | — | 129.6 ± 20.2 | 168.2 ± 24.8 | 142.8 ± 11.5 |
| MOLP-8 | 0 | 100 | 100 | 100 | — | — | — |
| | 1 | 112.9 ± 5.7 | 107.8 ± 0.5 | 116.1 ± 4.6 | — | — | — |
| | 2.5 | 131.7 ± 13.4 | 119.3 ± 7.7 | 124.7 ± 13.5 | — | — | — |

The symbol “—” denotes no expression in this cell line.

The expression of MICA and MICB after treatment with tinostamustine was then investigated by RT-qPCR. In line with flow cytometry data, tinostamustine increased the expression of MICA at the mRNA level in the four MM cell lines tested, and that of MICB in all of them except for MOLP-8 (FIG. 7B).

The effect of tinostamustine on MICA and MICB expressed by MM patients' plasma cells was further investigated ex vivo. In 6 out of the 9 patients analyzed (p2188, p2249, p2316, p2400, p2483 and p2485) the expression of MICA in tinostamustine-treated cells was at least twice than that in DMSO-treated cells (FIG. 7C). In the case of MICB, its expression increased by more than 1.5-fold after treatment with tinostamustine in 3 out of 6 the patients analyzed (p2400, p2483 and p2485).

ULBP1, another NKG2D ligand, was not basally expressed in any of the MM cell lines tested and did not change after treatment with tinostamustine (data not shown). In the case of ULBP2 and ULBP3 (also NKG2D ligands) and CD155 and CD112 (DNAM-1 ligands) results were heterogeneous among different MM cell lines as shown in Table 2 below.

TABLE 2
Effect of tinostamustine on the expression of several
NKG2D and DNAM-1's ligands by flow cytometry

| | EDO-S101 | | | | |
|---|---|---|---|---|---|
| | (μM) | ULBP2 | ULBP3 | CD155 | CD112 |
| JJN3 | 0 | 100 | — | 100 | — |
| | 1 | 110.5 ± 9.5 | — | 161.4 ± 14.3 | — |
| | 2.5 | 112.9 ± 13.4 | — | 156.8 ± 24.7 | — |
| MM.1S | 0 | 100 | — | 100 | — |
| | 1 | 95.8 ± 4.5 | — | 113.3 ± 3.9 | — |
| | 2.5 | 96.3 ± 12.3 | — | 111.5 ± 15.3 | — |
| RPMI-8226 | 0 | — | 100 | 100 | 100 |
| | 1 | — | 111.7 ± 14.8 | 123.9 ± 6.1 | 116.6 ± 13.2 |
| | 2.5 | — | 129.6 ± 20.2 | 168.2 ± 24.8 | 142.8 ± 11.5 |
| MOLP-8 | 0 | 100 | — | — | — |
| | 1 | 116.1 ± 4.6 | — | — | — |
| | 2.5 | 124.7 ± 13.5 | — | — | — |

MFI levels of the indicated NKG2D and DNAM-1's ligands normalized to isotype control after treatment with tinostamustine (1 and 2.5 μM) for 48 hours. Results are shown as the percentage with respect to DMSO-treated cells that were considered as 100%. Expression levels correspond to the average of three experiments.

Thus, among the cell lines intrinsically expressing NKG2D ligands, a trend towards an increase was observed (except ULBP2 in MM.1S).

### Example 4—EDO-S101 Increases the Efficacy of Daratumumab in MM Cell Lines

Without wishing to be bound by theory, it is thought that daratumumab acts through four different mechanisms to kill cancer cells: (i) cross-linking mediated apoptosis (ii) complement dependent cytotoxicity (CDC), (iii) Antibody-Dependent Cellular Cytotoxicity (ADCC) and (iv) Antibody-Dependent Cellular Phagocytosis (ADCP).

The effect of treatment of MM cells with EDO-S101 on the efficacy of daratumumab was investigated for each mechanism of action (i)-(iv). In each case (i)-(iv), the MM cells (MM1.S, RPMI-8226, MOLP-8) were either untreated (0 μM EDO-S101) or treated with EDO-S101 (2.5 μM) in accordance with the general methods outlined above.

### (i) Cross-Linking Mediated Apoptosis

Since the above data showed that tinostamustine upregulated CD38 and MICA/B expression in different myeloma cell lines, this effect was expected to translate into an improvement of daratumumab mediated ADCC.

MM cells (MOLP-8 cells pre-treated with 0, 0.1, 0.5, 1 or 2.5 μM EDO-S101 for 48 hours in accordance with the methods outlined above) were incubated for 24 hours in the presence of 1 μg/mL daratumumab or the corresponding isotype control, along with 10 μg/mL F(ab)₂ fragments for cross-linking. Apoptosis was evaluated by flow cytometry in accordance with the methods outlined above, using AnnexinV/7AAD staining. The effect of EDO-S101 on the crosslinking-mediated apoptosis induced by daratumumab was assessed using the following formula:

$$Daratumumab mediated apoptosis ( \% ) = 100 - [ \frac{( 100 - treatment death ) * 100}{( 100 - control death )} ]$$

“Treatment death” refers to the proportion of MM cells that were treated with daratumumab (as a percentage) that were determined to have undergone apoptosis. “Control death” refers to the basal death of the ESO-S101 pre-treated or non pre-treated myeloma cells, respectively of daratumumab. This calculation was performed for MM cells treated with each concentration of EDO-S101 investigated (0, 0.1, 0.5, 1 and 2.5 μM).

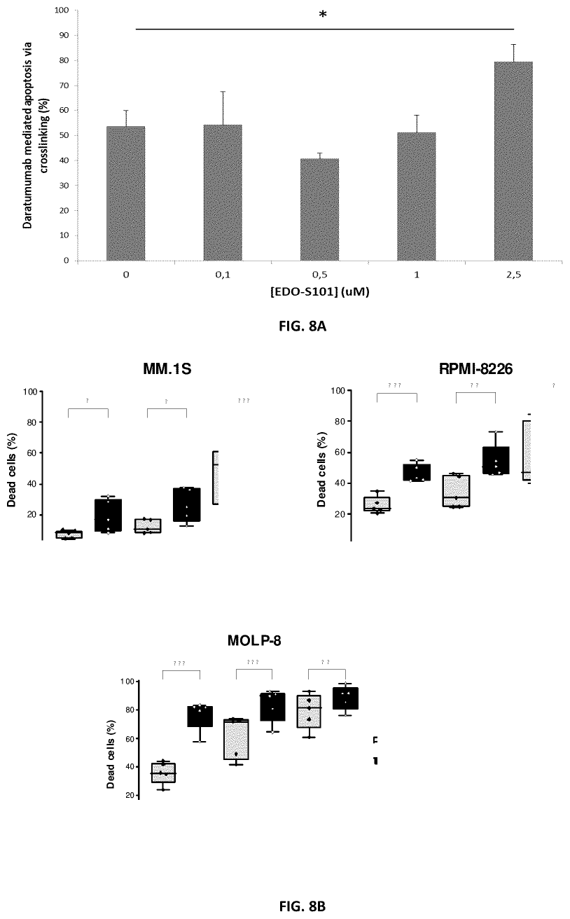

As shown in FIG. 8A (each bar shows mean±SEM (n=3). *p<0.05 by the Student t test.), daratumumab-mediated apoptosis was found to be higher in MM (MOLP-8) cells pre-treated with EDO-S101 (2.5 μM), relative to control cells not treated with EDO-S101 (p=0.0454).

Similarly, pre-treatment of other MM cell lines (MM.1S and RPMI-8226 cells, in addition to MOLP-8 cell) with tinostamustine (2.5 μM) for 48 hours also significantly increased the percentage of dead myeloma cells in the presence of NK cells and absence of daratumumab (NK-cell direct cytotoxicity) when compared to DMSO-pretreated cells (FIG. 8B). Similarly, the percentage of dead myeloma cells was higher in tinostamustine-pretreated myeloma cells vs DMSO-pretreated myeloma cells when they were exposed to NK cells in the presence of daratumumab (daratumumab-dependent cellular cytotoxicity or ADCC; FIG. 8B).

These data indicate that pre-treatment of MM cells with EDO-S101 significantly increases daratumumab-mediated apoptosis in MM cells.

### (ii) Complement-Dependent Cytotoxicity (CDC)

MM cells (MOLP-8 pre-treated with 0 or 2.5 μM EDO-S101 for 48 hours in accordance with the methods outlined above) were incubated with 0.1 μg/mL daratumumab, 1 μg/ml daratumumab, or the corresponding isotype control, for 30 minutes at 37° C. 10% human serum was then added as source of complement and the cells were incubated for 4 hours. The percentage of dead cells was then evaluated by flow cytometry in accordance with the methods outlined above, using AnnexinV/7AAD staining.

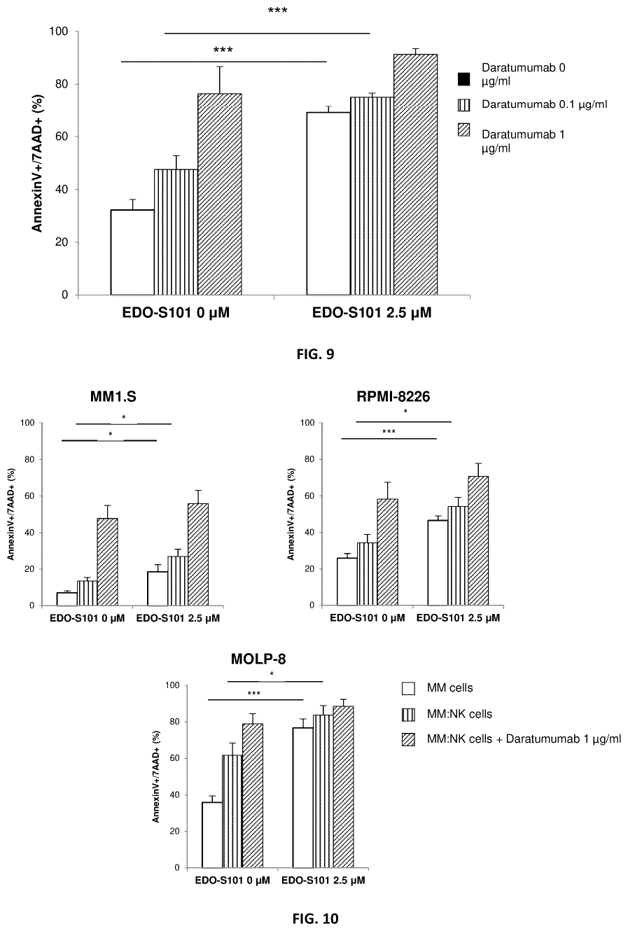

As shown in FIG. 9 (each bar shows mean±SEM (n=5). ***p<0.001 by the Student t test) pre-treatment of MM cells with EDO-S101 (2.5 μM) enhanced daratumumab mediated CDC.

These data indicate pre-treatment of MM cells with EDO-S101 significantly increases daratumumab-mediated complement-dependent cytotoxicity (CDC) in MM cells.

(iii) ADCC (Antibody-Dependent Cellular Cytotoxicity)

MM cells (MM1.S, RPMI-8226, MOLP-8 cells pre-treated with 0 or 2.5 μM EDO-S101 in accordance with the methods outlined above) were co-cultured with NK-cells (ratio 1:1), in the presence of daratumumab (1 μg/mL) or the corresponding isotype control, for 4 hours at 37° C. MM cells cultured in the absence of NK-cells and in the absence of daratumumab were also investigated as a control. ADCC was then evaluated using flow cytometry in accordance with the methods outlined above, using the following panel: CD38me-FITC/AnnexinV-PE/7AAD/CD45-APC.

FIG. 10 (each bar shows mean±SEM [MM1.S n=6; RPMI-8226 n=9; MOLP-8 n=5]. *p<0.05, **p<0.01 and ***p<0.001 by the Student t test) demonstrates that daratumumab-mediated ADCC was significantly enhanced in all MM cell lines pre-treated with EDO-S101 (MM:NK cells+daratumumab), relative to cell lines that were not pre-treated with EDO-S101 (MM cells).

Moreover, FIG. 10 demonstrates that pre-treatment of MM cell lines with EDO-S101 significantly enhances direct cytotoxicity mediated by NK cells in the absence of daratumumab (MM:NK cells), relative to MM cells (MM cells). In view of the observed increase in NK-cell mediated cytotoxicity, these data demonstrate that pre-treatment of MM cells with EDO-S101 increases the immunogenicity of MM cells, which in turn, would lead to further tumor death.

The data in FIG. 10 provides further evidence that pre-treatment of MM cells with EDO-S101 enhances NK-mediated cytotoxicity in MM cell lines, as well as increasing anti-CD38 antibody mediated ADCC.

### (iv) ADCP (Antibody-Dependent Cellular Phagocytosis)

Mononuclear cells were isolated from buffy coats from healthy donors using density gradient centrifugation with Ficoll and subsequently CD14+ monocytes were magnetically isolated in an autoMACS® Pro Separator. Monocytes were cultured for 7 days in the presence of GM-CSF (10 ng/mL) to induce differentiation to macrophages. For the evaluation of ADCP, myeloma cells were either untreated or pre-treated with 2.5 μM EDO-S101 for 48 hours. Cells were washed to remove EDO-S101 from the culture medium, and untreated and pre-treated myeloma cells were stained with PKH26. Co-cultures of macrophages and myeloma cells^{PKH26+} (ratio 3:1) were incubated with different concentrations of daratumumab or the corresponding isotype control for 4 hours at 37° C. ADCP was evaluated by flow cytometry with the panel: CD11b-FITC/PKH26/CD138-APC. Macrophages that have phagocytosed MM cells are double positive (CD11b₊/PKH26₊) whereas triple positives (CD11b₊/PKH26₊/CD138₊) are considered as macrophages only adhering to target cells (not engulfing them).

ADCP was quantified as the percentage of double positive events using the following formula:

$$Double positive events ( \% ) = 100 - [ \frac{( 100 - treatment double positive ) * 100}{( 100 - control double positive )} ]$$

‘Treatment double positive cells’ refers to the proportion of macrophages (as a percentage) determined to have phacocytosed MM cells in co-cultures treated with daratumumab. ‘Control double positive cells’ refers to the proportion of macrophages (as a percentage) determined to have phacocytosed MM cells in co-cultures that were ED-S101 untreated or pre-treated but not treated with daratumumab.

Elimination of myeloma cells was calculated using the following formula:

$$Eliminated myeloma cells ( \% ) = 100 - [ \frac{( Remaining myeloma cells with treatment )}{( Remaining myeloma cells without treatment )} * 100 ]$$

“myeloma cells without treatment” are the remaining myeloma cells (either untreated or previously pretreated with EDO-S101) in the absence of daratumumab.

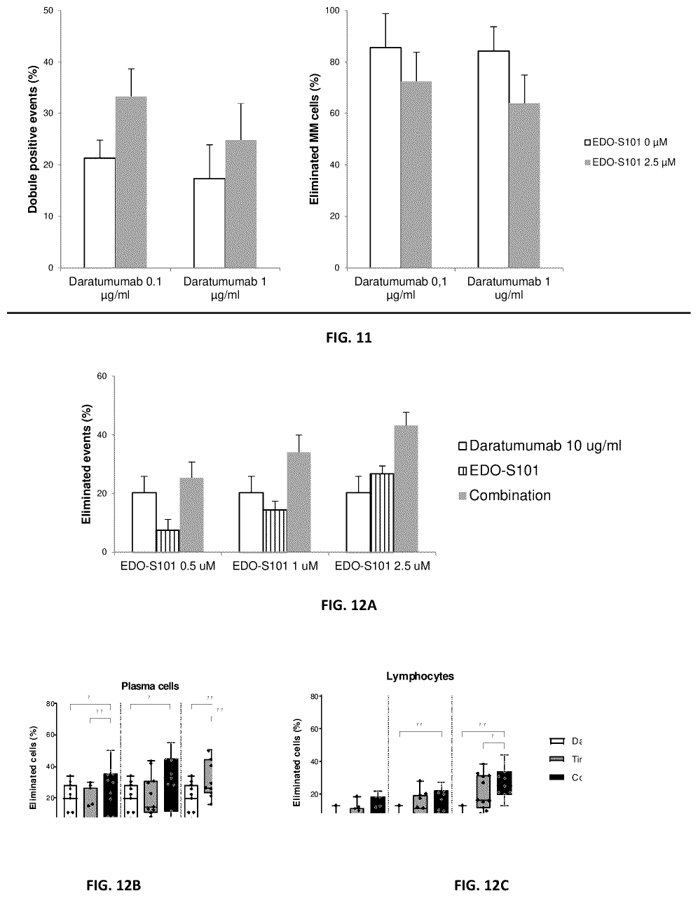

As shown in FIG. 11, the percentage of double positive events (macrophages that have phagocytosed myeloma cells) was increased in co-cultures comprising MM cells pre-treated with EDO-S101, as compared to co-cultures comprising MM cells that were not treated with EDO-S101 (FIG. 11; each bar shows mean±SEM (n=3). Furthermore, as also shown in FIG. 11, the percentage of eliminated MM cells was decreased in cell lines pre-treated with EDO-S101.

These data indicate that treatment of MM cells with EDO-S101 increases the efficacy of daratumumab-mediated ADCP.

### Example 5—Combination of EDO-S101 and Daratumumab Ex Vivo

The effect of the combination of EDO-S101+daratumumab was also evaluated in ex vivo cultures of freshly isolated bone marrow samples from 10 patients with MM.

Bone marrow (BM) samples from 10 patients with multiple myeloma were lysed to isolate plasma cells and lymphocytes (i.e., remove red blood cells). The isolated cells were cultured in RPMI-1640+20% FBS for 24 hours, in the presence of either daratumumab (10 μg/mL), EDO-S101 (0.5, 1 and 2.5 M), or a combination of daratumumab (10 μg/mL)+EDO-S101 (0.5, 1 or 2.5 μM). As a control, isolated cells were also cultured with a corresponding isotype control to daratumumab; and with a combination of the corresponding isotype control and EDO-S101.

Cells were collected in BD Trucount™ Absolute Counting Tubes (Becton Dickinson, Franklin Lakes, NJ) and stained with the corresponding antibodies (CD38/CD45/CD56) for flow cytometry analysis.

The percentage of eliminated cells was calculated using the following formula, and following manufacturer instructions on myelomatous plasma cells (CD38^{+bright}, CD45^{-/low}, SSC^{low/intermediate}, CD56^{-/+}) and normal lymphocytes (CD45⁺⁺, SSC^{low}) populations:

$$Eliminated myeloma cells ( \% ) = 100 - [ \frac{( Remaining myeloma cells with treatment )}{( Remaining myeloma cells without treatment )} * 100 ]$$

‘Remaining myeloma cells with treatment’ refers to the proportion of MM cells (as a percentage) determined to be viable in cultures treated with daratumumab. ‘Remaining myeloma cells without treatment’ refers to the proportion of MM cells (as a percentage) determined to be viable in cultures that were not treated with daratumumab.

The results showed that the percentage of eliminated cells was significantly higher with the double combination than with individual treatments (FIGS. 12A and 12B) with an acceptable toxicity of the combination on heathy lymphocytes, slightly increased for the highest doses of tinostamustine (FIG. 12C).

These data indicate that a combination of daratumumab and EDO-S101 is efficacious in killing MM cells and therefore, in the treatment of multiple myeloma, and moreover that said combination enhances the efficacy of daratumumab in treating MM cells.

### Example 6—Combination of EDO-S101+Daratumumab In Vivo

The in vivo effects of treatment with EDO-S101+daratumumab in a murine model of disseminated human myeloma was evaluated.

### (i) CB17-SCID Mice Bearing MM1S-Luc Derived Tumors

CB17-SCID mice bearing MM1S-luc derived tumors were treated with EDO-S101, daratumumab, a combination thereof, or vehicle, in accordance with the methods outlined above. MM1S-Luc cells (3×10⁶) were intravenously injected into CB17-SCID mice. After 2 weeks, mice were randomized into four groups: one receiving vehicle (i.v., weekly), another group receiving EDO-S101 (15 mg/kg i.v., weekly on Tuesdays), another group receiving daratumumab (8 mg/kg i.p., weekly on Wednesdays) and the last group received the double combination of EDO-S101+daratumumab. Survival of mice analyzed with a Kaplan-Meier curve. Both drugs were administered over a period of 4 weeks.

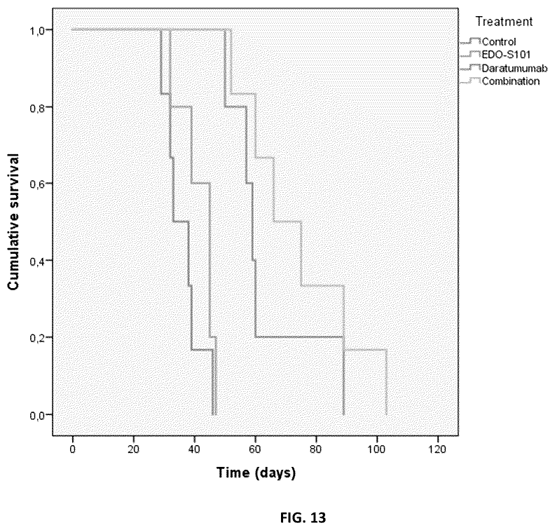

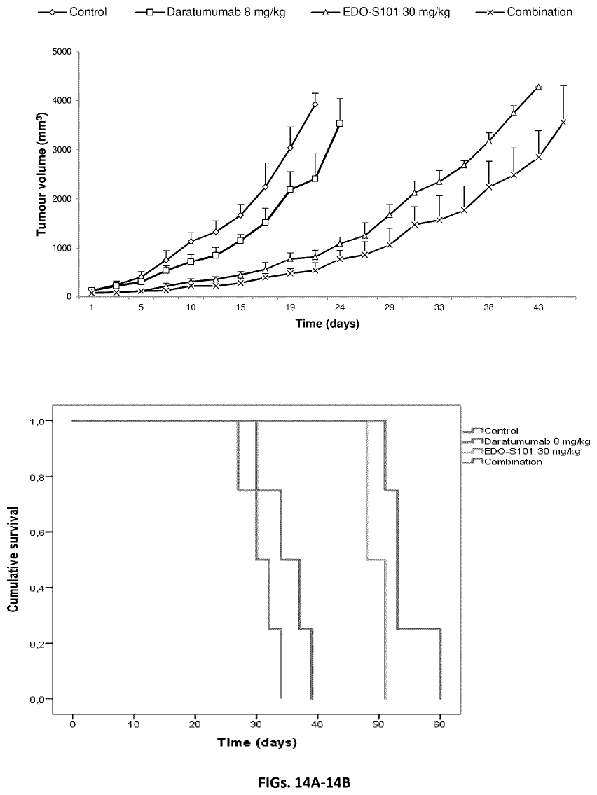

The median survival of mice treated with a combination of daratumumab and EDO-S101 combination (median survival was 66 days; range, 48 to 84 days) was increased relative to mice treated with daratumumab (median survival 59 days; range, 55 to 63 days) or EDO-S101 (median survival 45 days; range, 40 to 51 days) as monotherapies (FIG. 13). The control cohort receiving a vehicle (median survival 33 days; range, 26 to 40 days) demonstrated the poorest survival outcome.

These data indicate that a combination of EDO-S101 and daratumumab increases overall survival relative to EDO-S101 or daratumumab as monotherapies.

(ii) First Subcutaneous Plasmacytoma Model with NSG Mice

NSG mice bearing a subcutaneous plasmacytoma derived from MM.1S cells were randomized and treated with a vehicle, Daratumumab, EDO-S101 or a combination thereof, in accordance with the methods outlined above. EDO-S101 was administered on Mondays and human NK cells isolated from buffy coats from healthy donors and daratumumab on Tuesdays.

Mice treated with a combination of daratumumab and EDO-S101 exhibited significant delayed tumor growth relative to mice treated with daratumumab (p=0.011) or EDO-S101 (p=0.028) as monotherapies (FIG. 14A). In addition, mice treated with the combination exhibited increased median survival (53 days±0.87) relative to mice treated with daratumumab or EDO-S101 as monotherapies (34 days±3.5; 48 days±NE, respectively) or mice treated with a vehicle (30 days±2.5) (FIG. 14B; Kaplan-Meier curve). Statistical differences in survival were calculated between combination vs vehicle (p=0.007), combination vs EDO-S101 (p=0.028) and combination vs daratumumab (p=0.007).

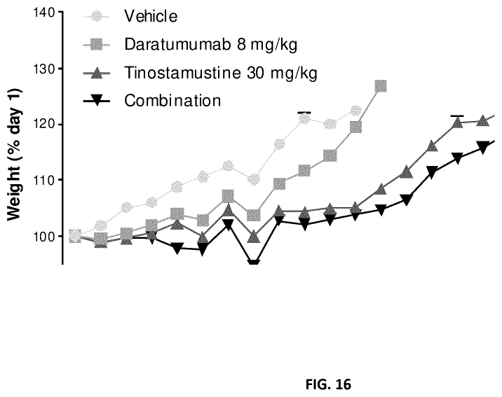

All treatments were generally well tolerated with less than 10% body weight loss (FIG. 16).

These data indicate that a combination of EDO-S101 and daratumumab is effective in delaying tumor growth and therefore progression-free survival, and moreover in improving overall survival, relative to EDO-S101 or daratumumab as monotherapies.

(iii) Second Subcutaneous Plasmacytoma Model with CB17-SCID Mice

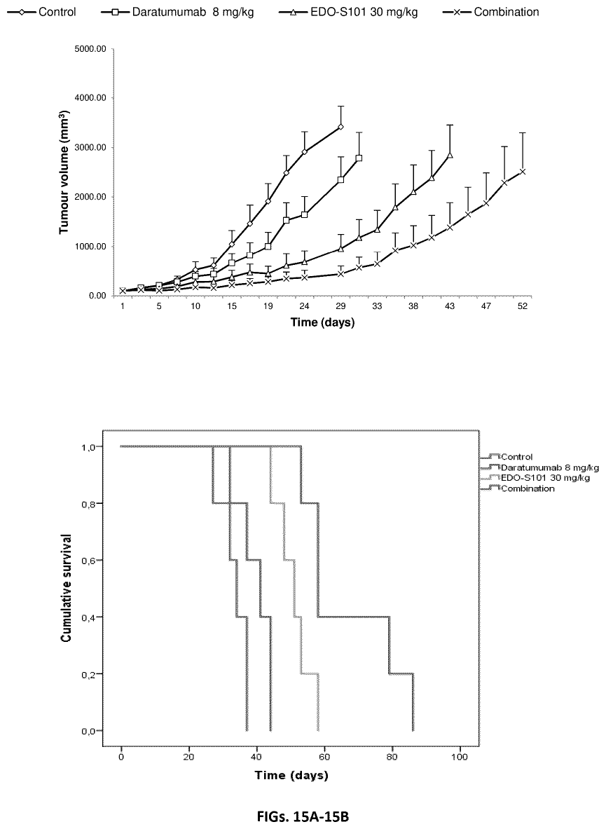

In the second xenograft plasmacytoma model, CB17-SCID mice bearing a subcutaneous plasmacytoma derived from MM1.S cells were randomized to receive vehicle, daratumumab, EDO-S101 or the double combination. Mice were treated with EDO-S101 on Mondays and daratumumab was administered on Tuesdays. Mice treated with a combination of daratumumab and EDO-S101 exhibited delayed tumor growth relative to daratumumab as a monotherapy or EDO-S101 (FIG. 15A). In addition, mice treated with the combination exhibited increased overall median survival (58 days±2.739) relative to mice treated with daratumumab (41 days±4.382, p=0.003) or EDO-S101 (51 days±3.286; p=0.028) as monotherapies (FIG. 15B). Furthermore, statistical differences in survival were also found between the combination vs vehicle (58 days±2.739 vs 34 days±2.191 days, respectively; p=0.003).

Similarly to the NK cell-humanized NSG model, all treatment schemes were generally well tolerated with less than 5% body weight loss (data not shown).

These data indicate that a combination of EDO-S101 and daratumumab is effective in delaying tumor growth, and moreover in improving overall survival, relative to EDO-S101 or daratumumab as monotherapies.

In summary, these examples show that EDO-S101 increases CD38 surface expression, which in turn is translated into increased daratumumab binding to myeloma cells. Furthermore, EDO-S101 increases the expression of a number of ligands for the NK cell activating receptors NKG2D and DNAM-1. As explained above, both the increase of CD38 surface expression as well as the increase in expression of ligands for NK cell activating receptors induced by EDO-S101, are translated into a higher tumor sensitivity to a direct attack by NK cells and increased efficacy of ADCC mediated by daratumumab. Furthermore, the results show a statistically significant increase in apoptosis via crosslinking and the CDC mediated by daratumumab when myeloma cells were pre-treated with EDO-S101.

In ex vivo experiments with patient samples, it was observed that treatment with the double combination of EDO-S101+daratumumab induced a significantly higher percentage of eliminated myeloma cells than the individual treatments.

In vivo data demonstrated that pre-treatment treatment with EDO-S101 followed by daratumumab controlled tumor growth and improved the survival of mice compared to those treated with daratumumab or EDO-S101 as a monotherapy.

All these data demonstrates that combination therapy using EDO-S101 (tinostamustine) and anti-CD38 monoclonal antibody (such as daratumumab) has a synergistic effect to treat multiple myeloma compared to monotherapies using tinostamustine or anti-CD38 monoclonal antibody (such as daratumumab) alone.

1. A method of treating cancer in a patient in need thereof, comprising administering to the patient a therapeutically effective amount of tinostamustine or a pharmaceutically acceptable salt thereof, in combination with a therapeutically effective amount of one or more anti-CD38 antibod(ies) or an antigen-binding fragment thereof, wherein the cancer is capable of expressing CD38 upon contacting tinostamustine or pharmaceutically acceptable salt thereof.

2. The method of claim 1, wherein the one or more anti-CD38 antibodies are selected from daratumumab, isatuximab, MOR202 or combinations thereof.

3. The method of claim 1, wherein the one or more anti-CD38 antibodies are daratumumab.

4. The method of claim 1, wherein the cancer is a haematological cancer.

5. The method of claim 4, wherein the haematological cancer is selected from the group consisting of leukaemia, lymphoma and multiple myeloma.

6. The method of claim 5, wherein the haematological cancer is multiple myeloma.

7. The method of claim 1, wherein tinostamustine or a pharmaceutically acceptable salt thereof and the one or more anti-CD38 antibodies are administered concurrently, sequentially, or separately.

8. The method of claim 7, wherein tinostamustine or pharmaceutically acceptable salt thereof, and the one or more anti-CD38 antibodies, are administered separately, and wherein tinostamustine or pharmaceutically acceptable salt thereof is optionally administered prior to the one or more anti-CD38 antibodies.

9. The method of claim 7, wherein tinostamustine or pharmaceutically acceptable salt thereof, and the one or more anti-CD38 antibodies, are administered concurrently.

10. The method of claim 1, wherein the molar ratio of tinostamustine or pharmaceutically acceptable salt thereof, and the one or more anti-CD38 antibodies, is 1:1000 to 1000:1, 1:700 to 700:1, 1:500 to 500:1, 1:300 to 300:1, 1:100 to 100:1, or 1:50 to 50:1.

11. The method of claim 1, wherein the expression of CD38 in the cancer or cancer cells is heterogeneous.

12. The method of claim 1, wherein the expression of CD38 in the cancer or cancer cells is low.

13. A combination comprising (a) tinostamustine or a pharmaceutically acceptable salt thereof, and (2) one or more anti-CD38 antibodies or an antigen-binding fragment thereof.

14. The combination of claim 13, wherein the one or more anti-CD38 antibodies are selected from the group consisting of daratumumab, isatuximab, MOR202, and combinations thereof.

15. The combination of claim 13, wherein the one or more anti-CD38 antibodies are daratumumab.

16. The combination of claim 13, wherein the molar ratio of Tinostamustine or pharmaceutically acceptable salt thereof, and the one or more anti-CD38 antibodies, is 1:1000 to 1000:1, 1:700 to 700:1, 1:500 to 500:1, 1:300 to 300:1, 1:100 to 100:1, or 1:50 to 50:1.

17. A pharmaceutical composition comprising a pharmaceutically acceptable carrier and a combination according to claim 13.

18. A kit comprising a combination according to claim 13, or a pharmaceutical composition according to claim 17.

19. The kit according to claim 18, further comprising instructions for treating a cancer in a patient.
